(19) United States
(12) Patent Application Publication
(10) Pub. No.: US 2026/0083875 A1
(43) Pub. Date: Mar. 26, 2026

(54) MEDICAL DEVICE THAT INCLUDES A RHENIUM-CHROMIUM ALLOY

(71) Applicant: MiRus LLC, Marietta, GA (US)

(72) Inventor: Noah Roth, Marietta, GA (US)

(21) Appl. No.: 19/401,454

(22) Filed: Nov. 26, 2025

Related U.S. Application Data
(63) Continuation of application No. 18/116,677, filed on Mar. 2, 2023.
(60) Provisional application No. 63/316,077, filed on Mar. 3, 2022.

(51) Int. Cl. A61L 27/04; A61F 2/24; A61F 2/90; A61L 27/06; A61L 27/34; A61L 27/50; A61L 27/54; C22C 27/00; C22C 27/06

(52) U.S. Cl. CPC A61L 27/047; A61F 2/2418; A61F 2/90; A61L 27/06; A61L 27/34; A61L 27/507; A61L 27/54; C22C 27/00; C22C 27/06; A61F 2240/001; A61F 2250/0067; A61L 2300/62; A61L 2430/20

(57) ABSTRACT

A rhenium-chromium metal alloy or rhenium alloy that can be used to at least partially form a medical device.

The present disclosure is a continuation of U.S. patent application Ser. No. 18/116,677 filed Mar. 2, 2023, which in turn claims priority on U.S. Provisional Application Ser. No. 63/316,077 filed Mar. 3, 2022, all of which are incorporated herein.

The disclosure relates generally to rhenium metal alloys, more particularly to rhenium-chromium metal alloys, and even more particularly to rhenium-chromium metal alloys that at least partially form a medical device.

### BACKGROUND OF DISCLOSURE

Stainless steel, cobalt-chromium alloys, and TiAlV alloys are some of the more common metal alloys used for medical devices. Although these alloys have been successful in forming a variety of medical devices, these alloys have several deficiencies.

Many cardiovascular devices such as stents, expandable heart valves, and the like are inserted into a patient via the vascular system of a patient and then expanded at the treatment site. These devices are typically crimped onto a catheter prior to insertion into a patient. The minimum diameter to which the cardiovascular device can be crimped onto the catheter will set a limit to the size of the cardiovascular passageway (e.g., blood vessel) to which the cardiovascular device can be inserted. Smaller crimp diameters can result in reduced damage to a blood vessel and/or organ (e.g., heart, etc.) when inserting into and/or placing the cardiovascular device at the treatment site. Smaller crimp diameters can also allow the cardiovascular device to be placed in smaller diameter blood vessels (e.g., blood vessels located in the brain, etc.).

The crimp diameter of the expandable cardiovascular device can be reduced by reducing the thickness and/or size of the frame, struts, etc., of the cardiovascular device. However, such reduction in size also affects the strength of the cardiovascular device after being expanded. After the cardiovascular device is expanded, it must retain its expanded shape at the treatment area, otherwise the cardiovascular device could become dislodged from the treatment area, could damage the treatment area, and/or fail to properly function at the treatment area. As such, cardiovascular devices formed of tradition materials such as stainless steel (e.g., 316L: 17-19 wt. % chromium, 13-15 wt. % nickel, 2-4 wt. % molybdenum, 2 wt. % max manganese, 0.75 wt. % max silicon, 0.03 wt. % max carbon, balance iron) and cobalt-chromium alloys (e.g., MP35N: 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt) are required to maintain a frame and/or strut size/thickness that limits how small of crimping diameter can be obtained by the crimped cardiovascular device. Other types of cobalt-chromium alloys that have been used are Phynox and Elgiloy alloy (38-42 wt. % cobalt, 18-22 wt. % chromium, 14-18 wt. % iron, 13-17 wt. % 5 nickel, 6-8 wt. % molybdenum), and L605 alloy (18-22 wt. % chromium, 14-16 wt. % W, 9-11 wt. % nickel, balance cobalt).

Also, traditional materials such as stainless steel (316L) and cobalt-chromium alloys (e.g., MP35N, etc.) have a degree of recoil after being crimped and expanded that can interfere with obtaining a minimum crimping diameter and/or can adversely affect the placement of the expandable cardiovascular device at a treatment area. During a crimping process, a crimping device is typically used to crimp the cardiovascular device onto a catheter. After an initial crimping process, tradition materials such as stainless steel and cobalt-chromium alloys recoil to a larger diameter by 9+% of the minimum crimped diameter. As such, the cardiovascular device must be crimped multiple times onto a catheter to attempt to obtain a smaller crimped diameter on the catheter. However, subjecting the cardiovascular device to multiple crimpings can result in damage to the cardiovascular device (e.g., damage to the frame and/or struts of the cardiovascular device, damage to leaflets on an expandable heart valve, etc.). Likewise, when the cardiovascular device is expanded at a treatment area, the traditional materials of the cardiovascular device will recoil 9+% of the maximum expanded diameter. As such, the inflatable balloon on the catheter must be pressurized multiple times to repeatedly expand the cardiovascular device at the treatment area to ensure proper expansion of the cardiovascular device. However, subjecting the cardiovascular device to multiple balloon expansions can result in damage to the cardiovascular device (e.g., damage or breakage of a frame and/or strut, etc.) and/or damage to the treatment area (e.g., rupture of blood vessel, tear and/or puncture of tissue of an organ, etc.).

When a medical device is inserted into a patient, it is typically desirable for the medical device to resist ionization and/or corrosion while in the patient so as to not subject the patient to metal ions and/or oxides from the metals used to form the medical device while in the patient. Excessive ion release from the medical device can be potentially adverse to the patient. Although traditional materials such as stainless steel (316L) and cobalt-chromium alloys (e.g., MP35N, etc.) are very stable when inserted into patients, some degree of metal ion release occurs when the medical device is in the patient.

In view of the current state of the art of medical devices, there is a need for an improved medical device that a) produces less recoil compared to medical devices formed of stainless steel, cobalt-chromium alloys, or TiAlV alloys, b) can form smaller crimping diameters compared to medical devices formed of stainless steel, cobalt-chromium alloys, or TiAlV alloys, and/or c) has reduced metal ion release compared to medical devices formed of stainless steel, cobalt-chromium alloys, or TiAlV alloys.

### SUMMARY OF THE DISCLOSURE

The present disclosure is direct to rhenium-chromium metal alloy and a medical device that is at least partially made of a rhenium-chromium metal alloy or rhenium alloy. The medical device that is at least partially formed of the rhenium-chromium metal alloy or rhenium alloy can include an orthopedic device, PFO (patent foramen ovale) device, stent, valve (e.g., heart valve, TAVR valve, mitral valve replacement, tricuspid valve replacement, pulmonary valve replacement, etc.), spinal implant, frame and other structures for use with a spinal implant, vascular implant, graft, guide wire, sheath, catheter, needle, stent catheter, electrophysiology catheter, hypotube, staple, cutting device, any type of implant, pacemaker, dental implant, dental crown, dental braces, wire used in medical procedures, bone implant, artificial disk, artificial spinal disk, prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage, bone plate nail, rod, screw, post, cage, plate, pedicle screw, cap, hinge, joint system, anchor, spacer, shaft, anchor, disk, ball, tension band, locking connector other structural assembly that is used in a body to support a structure, mount a structure, and/or repair a structure in a body such as, but not limited to, a human body, animal body, etc. In one non-limiting embodiment, the medical device includes an expandable frame (e.g., stent, prosthetic heart valve, etc.) that can plastically deform radially outwardly by an expansion arrangement (e.g., inflatable balloon, etc.). In another non-limiting embodiment, the rhenium-chromium metal alloy or rhenium alloy is not a self-expanding alloy.

In accordance with another and/or alternative non-limiting aspect of the present disclosure, there is provided a medical device partially or fully formed of a rhenium-chromium metal alloy or rhenium alloy. In one non-limiting embodiment, 5-100% (and all values and ranges therebetween) of the medical device is formed of the rhenium-chromium metal alloy or rhenium alloy.

In accordance with another and/or alternative non-limiting aspect of the present disclosure, at least 50 wt. % (e.g., 50-75 wt. % and all values and ranges therebetween) of the rhenium-chromium metal alloy or rhenium alloy includes rhenium. In another non-limiting embodiment, at least 55 wt. % of the rhenium-chromium metal alloy or rhenium alloy includes rhenium. In another non-limiting embodiment, at least 60 wt. % of the rhenium-chromium metal alloy or rhenium alloy includes rhenium.

In accordance with another and/or alternative non-limiting aspect of the present disclosure, at least 25 wt. % (e.g., 25-49.9 wt. % and all values and ranges therebetween) of the rhenium-chromium metal alloy or rhenium alloy includes chromium. In another non-limiting embodiment, at least 30 wt. % of the rhenium-chromium metal alloy or rhenium alloy includes chromium. In another non-limiting embodiment, at least 33 wt. % of the rhenium-chromium metal alloy or rhenium alloy includes chromium.

In accordance with another and/or alternative non-limiting aspect of the present disclosure, at least 50 wt. % (e.g., 50-74.9 wt. % and all values and ranges therebetween) of the rhenium-chromium metal alloy or rhenium alloy includes rhenium, at least 25 wt. % (e.g., 25-49.9 wt. % and all values and ranges therebetween) of the rhenium-chromium metal alloy or rhenium alloy includes chromium, and 0.1-25 wt. % (and all values and ranges therebetween) of the rhenium-chromium metal alloy or rhenium alloy includes one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium.

In accordance with another and/or alternative non-limiting aspect of the present disclosure, at least 55 wt. % (e.g., 55-69.9 wt. % and all values and ranges therebetween) of the rhenium-chromium metal alloy or rhenium alloy includes rhenium, at least 30 wt. % (e.g., 30-44.9 wt. % and all values and ranges therebetween) of the rhenium-chromium metal alloy or rhenium alloy includes chromium, and 0.1-15 wt. % (and all values and ranges therebetween) of the rhenium-chromium metal alloy or rhenium alloy includes one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium.

In accordance with another and/or alternative non-limiting aspect of the present disclosure, at least 60 wt. % (e.g., 60-69.9 wt. % and all values and ranges therebetween) of the rhenium-chromium metal alloy or rhenium alloy includes rhenium, at least 30 wt. % (e.g., 30-39.9 wt. % and all values and ranges therebetween) of the rhenium-chromium metal alloy or rhenium alloy includes chromium, and 0.1-10 wt. % (and all values and ranges therebetween) of the rhenium-chromium metal alloy or rhenium alloy includes one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium.

In accordance with another and/or alternative non-limiting aspect of the present disclosure, at least 62 wt. % (e.g., 62-67.9 wt. % and all values and ranges therebetween) of the rhenium-chromium metal alloy or rhenium alloy includes rhenium, at least 32 wt. % (e.g., 32-32.9 wt. % and all values and ranges therebetween) of the rhenium-chromium metal alloy or rhenium alloy includes chromium, and 0.1-6 wt. % (and all values and ranges therebetween) of the rhenium-chromium metal alloy or rhenium alloy includes one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium.

In accordance with another and/or alternative non-limiting aspect of the present disclosure, a weight percent of the rhenium in the rhenium-chromium metal alloy or rhenium alloy is less than 50 wt. %; and wherein the rhenium-chromium metal alloy or rhenium alloy includes 0.1-50 wt. % alloying agent; and wherein the alloying agent including one or more metals selected from a group consisting of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium; and wherein a weight percent of the chromium in the rhenium-chromium metal alloy or rhenium alloy is at least 25 wt. %; and a combined weight percent of the rhenium and the chromium is at least 35 wt. % (e.g., 35-99.9 wt. % and all values and ranges therebetween) of the rhenium-chromium metal alloy or rhenium alloy.

In accordance with another and/or alternative non-limiting aspect of the present disclosure, a weight percent of the rhenium in the rhenium-chromium metal alloy or rhenium alloy is less than 50 wt. %; and wherein the rhenium-chromium metal alloy or rhenium alloy includes 0.1-30 wt. % alloying agent; and wherein the alloying agent including one or more metals selected from a group consisting of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium; and wherein a weight percent of the chromium in the rhenium-chromium metal alloy or rhenium alloy is at least 25 wt. %; and a combined weight percent of the rhenium and the chromium is at least 70 wt. % (e.g., 70-99.9 wt. % and all values and ranges therebetween) of the rhenium-chromium metal alloy or rhenium alloy.

In accordance with another and/or alternative non-limiting aspect of the present disclosure, a weight percent of the rhenium in the rhenium-chromium metal alloy or rhenium alloy is less than 50 wt. %; and wherein the rhenium-chromium metal alloy or rhenium alloy includes 0.1-25 wt. % alloying agent; and wherein the alloying agent including one or more metals selected from a group consisting of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium; and wherein a weight percent of the chromium in the rhenium-chromium metal alloy or rhenium alloy is at least 25 wt. %; and a combined weight percent of the rhenium and the chromium is at least 75 wt. % (e.g., 75-99.9 wt. % and all values and ranges therebetween) of the rhenium-chromium metal alloy or rhenium alloy.

In accordance with another and/or alternative non-limiting aspect of the present disclosure, the rhenium-based alloy contains 10-60 atomic weight percent (atw. %) Re (and all values and ranges therebetween) and containing one or more metals selected from the group consisting of Mo, Cr, Ta, Nb, Ti, and Zr. In one non-limiting embodiment, the Cr content in the rhenium-based alloy constitutes the first or second highest atomic weight percent component of the rhenium-based alloy. In another non-limiting embodiment, the Cr content in the rhenium-based alloy constitutes the first or second highest atomic weight percent component of the rhenium-based alloy, and the atomic weight percent of Cr in the rhenium-based alloy is greater than an atomic weight percent of Mo in the rhenium-based alloy.

In accordance with another and/or alternative non-limiting aspect of the present disclosure, the rhenium-based alloy comprises 0.5-50 atw. % Re (and all values and ranges therebetween) and Cr 0.5-70 atw. % (and all values and ranges therebetween). In one non-limiting embodiment, the rhenium-based alloy comprises 5-50 atw. % Re and Cr 5-70 atw. %. In another non-limiting embodiment, the rhenium-based alloy comprises 5-50 atw. % Re and Cr 5-70 atw. %, and 0-49.9 awt. % (and all values and ranges therebetween) of one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium. In another non-limiting embodiment, the rhenium-based alloy comprises 15-50 atw. % Re and Cr 25-70 atw. %, and 0-49.9 wt. % (and all values and ranges therebetween) of one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium. In another non-limiting embodiment, the rhenium-based alloy comprises 20-50 atw. % Re and Cr 25-70 atw. %, and 0-49.9 awt. % (and all values and ranges therebetween) of one or more of aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and/or zirconium. In another non-limiting embodiment, the atomic weight percent of the rhenium plus chromium in the rhenium-based alloy is at least 50 awt. % (e.g., 50-100 awt. % and all values and ranges therebetween). In another non-limiting embodiment, the atomic weight percent of the rhenium plus chromium in the rhenium-based alloy is at least 60 awt. % (e.g., 60-100 awt. % and all values and ranges therebetween). In another non-limiting embodiment, the atomic weight percent of the rhenium plus chromium in the rhenium-based alloy is at least 75 awt. % (e.g., 75-100 awt. % and all values and ranges therebetween).

In another non-limiting embodiment, the rhenium-chromium metal alloy or rhenium alloy includes 0-0.1 wt. % (and all values and ranges therebetween) of impurities (e.g., metals other than aluminum, bismuth, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, and zirconium).

Several non-limiting examples of the rhenium-chromium metal alloy or rhenium alloy that can be made in accordance with the present disclosure are set forth below:

| Ele- | | | | |
|---|---|---|---|---|
| ment/ | | | | |
| Wt. % | Ex. 1 | Ex. 2 | Ex. 3 | Ex. 4 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Al | 0-25% | 0-25% | 0-25% | 0-25% |
| Co | 0-25% | 0-25% | 0-25% | 0-25% |
| Cu | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Si | 0-25% | 0-25% | 0-25% | 0-25% |
| Ag | 0-25% | 0-25% | 0-25% | 0-25% |
| C | <0.06 | <0.06 | <0.06 | <0.06 |
| N | <0.06 | <0.06 | <0.06 | <0.06 |
| O | <0.06 | <0.06 | <0.06 | <0.06 |
| Ele- | | | | |
| ment/ | | | | |
| Wt. % | Ex. 5 | Ex. 6 | Ex. 7 | Ex. 8 |
| Re | 50-72% | 55-72% | 60-72% | 65-72% |
| Cr | 28-50% | 28-45% | 28-40% | 28-35% |
| Mo | 0-25% | 0-25% | 0-25% | 0-25% |
| Bi | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Al | 0-10% | 0-10% | 0-10% | 0-10% |
| Co | 0-10% | 0-10% | 0-10% | 0-10% |
| Cu | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Si | 0-10% | 0-10% | 0-10% | 0-10% |
| Ag | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |
| Ele- | | | | |
| ment/ | | | | |
| Wt. % | Ex. 9 | Ex. 10 | Ex. 11 | Ex. 12 |
| Re | 50-70% | 55-70% | 60-70% | 65-70% |
| Cr | 30-50% | 30-45% | 30-40% | 30-35% |
| Mo | 0-10% | 0-10% | 0-10% | 0-10% |
| Bi | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Al | 0-10% | 0-10% | 0-10% | 0-10% |
| Co | 0-10% | 0-10% | 0-10% | 0-10% |
| Cu | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Si | 0-10% | 0-10% | 0-10% | 0-10% |
| Ag | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |
| Ele- | | | | |
| ment/ | | | | |
| Wt. % | Ex. 13 | Ex. 14 | Ex. 15 | Ex. 16 |
| Re | 50-67.5% | 55-67.5% | 60-67.5% | 65-67.5% |
| Cr | 32.5-50% | 32.5-45% | 32.5-40% | 32.5-35% |
| Mo | 0-10% | 0-10% | 0-10% | 0-10% |
| Bi | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Al | 0-10% | 0-10% | 0-10% | 0-10% |
| Co | 0-10% | 0-10% | 0-10% | 0-10% |
| Cu | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Si | 0-10% | 0-10% | 0-10% | 0-10% |
| Ag | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |
| Ele- | | | | |
| ment/ | | | | |
| Wt. % | Ex. 17 | Ex. 18 | Ex. 19 | Ex. 20 |
| Re | 50-67.5% | 55-67.5% | 60-67.5% | 65-67.5% |
| Cr | 32.5-50% | 32.5-45% | 32.5-40% | 32.5-35% |
| Mo | 0-5% | 0-5% | 0-5% | 0-5% |
| Bi | 0-5% | 0-5% | 0-5% | 0-5% |
| Ir | 0-5% | 0-5% | 0-5% | 0-5% |
| Nb | 0-5% | 0-5% | 0-5% | 0-5% |
| Ta | 0-5% | 0-5% | 0-5% | 0-5% |
| V | 0-5% | 0-5% | 0-5% | 0-5% |
| W | 0-5% | 0-5% | 0-5% | 0-5% |
| Mn | 0-5% | 0-5% | 0-5% | 0-5% |
| Tc | 0-5% | 0-5% | 0-5% | 0-5% |
| Ru | 0-5% | 0-5% | 0-5% | 0-5% |
| Rh | 0-5% | 0-5% | 0-5% | 0-5% |
| Hf | 0-5% | 0-5% | 0-5% | 0-5% |
| Os | 0-5% | 0-5% | 0-5% | 0-5% |
| Cu | 0-5% | 0-5% | 0-5% | 0-5% |
| Ir | 0-5% | 0-5% | 0-5% | 0-5% |
| Ti | 0-5% | 0-5% | 0-5% | 0-5% |
| Y | 0-5% | 0-5% | 0-5% | 0-5% |
| Zr | 0-5% | 0-5% | 0-5% | 0-5% |
| Al | 0-5% | 0-5% | 0-5% | 0-5% |
| Co | 0-5% | 0-5% | 0-5% | 0-5% |
| Cu | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Ag | 0-5% | 0-5% | 0-5% | 0-5% |
| C | <0.06 | <0.06 | <0.06 | <0.06 |
| N | <0.06 | <0.06 | <0.06 | <0.06 |
| O | <0.06 | <0.06 | <0.06 | <0.06 |
| Ele- | | | | |
| ment/ | | | | |
| Wt. % | Ex. 21 | Ex. 22 | Ex. 23 | Ex. 24 |
| Re | 30-75% | 40-75% | 45-75% | 45-70% |
| Cr | 25-70% | 25-65% | 25-55% | 30-55% |
| Mo | 0-25% | 0-25% | 1-25% | 2-25% |
| Bi | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Al | 0-25% | 0-25% | 0-25% | 0-25% |
| Co | 0-25% | 0-25% | 0-25% | 0-25% |
| Cu | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Si | 0-25% | 0-25% | 0-25% | 0-25% |
| Ag | 0-25% | 0-25% | 0-25% | 0-25% |
| C | <0.06 | <0.06 | <0.06 | <0.06 |
| N | <0.06 | <0.06 | <0.06 | <0.06 |
| O | <0.06 | <0.06 | <0.06 | <0.06 |

In Examples 1-24, it will be appreciated that all of the above ranges include any value between the range and any other range that is between the ranges set forth above. Any of the above values that include the < symbol includes the range from 0 to the stated value and all values and ranges therebetween.

In accordance with another and/or alternative non-limiting aspect of the present disclosure, the total combined weight percent of the rhenium and chromium in the rhenium-chromium metal alloy or rhenium alloys of Examples 1-24 is at least 60 wt. % (e.g., 60-100 wt. % and all values and ranges therebetween).

In accordance with another and/or alternative non-limiting aspect of the present disclosure, the total combined weight percent of the rhenium and chromium in the rhenium-chromium metal alloy or rhenium alloys of Examples 1-24 is at least 75 wt. %.

In accordance with another and/or alternative non-limiting aspect of the present disclosure, the total combined weight percent of the rhenium and chromium in the rhenium-chromium metal alloy or rhenium alloys of Examples 1-24 is at least 90 wt. %.

In accordance with another and/or alternative non-limiting aspect of the present disclosure, the total combined weight percent of the rhenium and chromium in the rhenium-chromium metal alloy or rhenium alloys of Examples 1-24 is at least 95 wt. %.

In accordance with another and/or alternative non-limiting aspect of the present disclosure, the total combined weight percent of the rhenium and chromium in the rhenium-chromium metal alloy or rhenium alloys of Examples 1-24 is at least 98 wt. %.

In accordance with another and/or alternative non-limiting aspect of the present disclosure, the total combined weight percent of the rhenium and chromium in the rhenium-chromium metal alloy or rhenium alloys of Examples 1-24 is at least 99 wt. %.

In accordance with another and/or alternative non-limiting aspect of the present disclosure, the total combined weight percent of the rhenium and chromium in the rhenium-chromium metal alloy or rhenium alloys of Examples 1-24 is at least 99.5 wt. %.

In accordance with another and/or alternative non-limiting aspect of the present disclosure, there is provided a medical device (e.g., stent, prosthetic heart valve, etc.) that is configured to be radially collapsible to a collapsed or crimped state for introduction into the body on a delivery catheter and radially expandable to an expanded state for implanting the prosthetic heart valve at a desired location in the body (e.g., blood vessel, heart, ureter, bile duct, pancreatic duct, esophagus, lung, eyes, sinus, oral stent, etc.). The frame of the medical device can be formed of a plastically-expandable material that permits crimping of the frame to a smaller profile for delivery and expansion of the medical device using an expansion device such as the balloon of a balloon catheter. In accordance with one non-limiting embodiment, the frame of medical device is formed of 20-100 wt. % (and all values and ranges therebetween) of the rhenium-chromium metal alloys or rhenium alloys as discussed above.

In accordance with another and/or alternative non-limiting aspect of the present disclosure, there is provided a medical device including a frame that includes a plurality of angularly-spaced posts, vertically-extending posts, or struts. The posts can be interconnected via a lower row of circumferentially-extending struts and an upper row of circumferentially-extending struts. The struts can be arrangement in a variety of patterns (e.g., zig-zag pattern, saw-tooth pattern, triangular pattern, polygonal pattern, oval pattern, etc.). One or more of the posts and/or struts can have the same or different thicknesses and/or cross-sectional shape and/or cross-sectional area. In accordance with one non-limiting embodiment, the frame of medical device is formed of 20-100 wt. % (and all values and ranges therebetween) of the rhenium-chromium metal alloys or rhenium alloys as discussed above.

In accordance with another and/or alternative non-limiting aspect of the present disclosure, there is provided a medical device including a frame that can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the struts on the frame and/or to fill in the openings between the struts. In accordance with one non-limiting embodiment, the frame of medical device is formed of 20-100 wt. % (and all values and ranges therebetween) of the rhenium-chromium metal alloys or rhenium alloys as discussed above.

In accordance with another and/or alternative non-limiting aspect of the present disclosure, the rhenium-chromium metal alloy or rhenium alloys as discussed above used to form at least a portion of the medical device has one or more improved properties (e.g., strength, durability, hardness, biostability, bendability, coefficient of friction, radial strength, flexibility, tensile strength, tensile elongation, longitudinal lengthening, stress-strain properties, reduced recoil, radiopacity, heat sensitivity, biocompatibility, improved fatigue life, crack resistance, crack propagation resistance, reduced magnetic susceptibility, etc.), improved conformity when bent, less recoil, increased yield strength, improved fatigue ductility, improved durability, improved fatigue life, reduced adverse tissue reactions, reduced metal ion release, reduced corrosion, reduced allergic reaction, improved hydrophilicity, reduced toxicity, reduced thickness of metal component, improved bone fusion, and/or lower ion release into tissue. These one or more improved physical properties of the rhenium-chromium metal alloys or rhenium alloys as discussed above can be achieved in the medical device without having to increase the bulk, volume, and/or weight of the medical device and, in some instances, these improved physical properties can be obtained even when the volume, bulk, and/or weight of the medical device is reduced as compared to medical devices at least partially formed from traditional stainless steel, titanium alloy, or cobalt and chromium alloy materials.

In another and/or alternative non-limiting aspect of the present disclosure, the rhenium-chromium metal alloys or rhenium alloys as discussed above that are used to at least partially form the medical device can thus 1) increase the radiopacity of the medical device, 2) increase the radial strength of the medical device, 3) increase the yield strength and/or ultimate tensile strength of the medical device, 4) improve the stress-strain properties of the medical device, 5) improve the crimping and/or expansion properties of the medical device, 6) improve the bendability and/or flexibility of the medical device, 7) improve the strength and/or durability of the medical device, 8) increase the hardness of the medical device, 9) improve the recoil properties of the medical device, 10) improve the biostability and/or biocompatibility properties of the medical device, 11) increase fatigue resistance of the medical device, 12) resist cracking in the medical device and resist propagation of crack, 13) enable smaller, thinner, and/or lighter weight medical device to be made, 14) reduce the outer diameter of a crimped medical device, 15) improve the conformity of the medical device to the shape of the treatment area when the medical device is used and/or expanded in the treatment area, 16) reduce the amount of recoil of the medical device to the shape of the treatment area when the medical device is expanded in the treatment area, 17) increase yield strength of the medical device, 18) improve fatigue ductility of the medical device, 18) improve durability of the medical device, 19) improve fatigue life of the medical device, 20) reduce adverse tissue reactions after implant of the medical device, 21) reduce metal ion release after implant of the medical device, 22) reduce corrosion of the medical device after implant of the medical device, 23) reduce allergic reaction after implant of the medical device, 24) improve hydrophilicity of the medical device, 25) reduce thickness of metal component of medical device, 26) improve bone fusion with medical device, 27) lower ion release from medical device into tissue, 28) reduce magnetic susceptibility of the medical device when implanted in a patient, and/or 29) reduce toxicity of the medical device after implant of the medical device.

In another and/or alternative non-limiting aspect of the present disclosure, the medical device that includes the rhenium-chromium metal alloys or rhenium alloys as discussed above is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printed coatings, chemical vapor deposition, chemical polishing, cleaning, pickling, ion beam deposition or implantation, sputter coating, vacuum deposition, hot isostatic pressing (HIP), sintering, extruding, isostatic pressing, vacuum sintering, etc. In one non-limiting embodiment, at least a portion or all of the medical device is formed by a 3D printing process.

In another and/or alternative non-limiting aspect of the present disclosure, the rhenium-chromium metal alloys or rhenium alloys as discussed above that is used to at least partially form the medical device optionally has a generally uniform density throughout the rhenium-chromium metal alloys or rhenium alloys, and also results in the desired yield and ultimate tensile strengths of the rhenium-chromium metal alloy or rhenium alloy. The density of the rhenium-chromium metal alloy is generally at least about 5 gm/cc (e.g., 5 gm/cc-21 gm/cc and all values and ranges therebetween), typically about 8-20 gm/cc, and typically at least about 9-19 gm/cc. This substantially uniform high density of the rhenium-chromium metal alloy or rhenium alloy can be used to improve the radiopacity of the rhenium-chromium metal alloy or rhenium alloy.

In another and/or alternative non-limiting aspect of the present disclosure, the rhenium-chromium metal alloys or rhenium alloys as discussed above optionally include a certain amount of carbon and oxygen; however, this is not required. These two elements have been found to affect the forming properties and brittleness of the rhenium-chromium metal alloys or rhenium alloys. The controlled atomic ratio of carbon and oxygen of the rhenium-chromium metal alloys or rhenium alloys also minimize the tendency of the rhenium-chromium metal alloys or rhenium alloys to form micro-cracks during the forming of the rhenium-chromium metal alloys or rhenium alloys at least partially into a medical device, and/or during the use and/or expansion of the medical device in a body. The control of the atomic ratio of carbon to oxygen in the rhenium-chromium metal alloys or rhenium alloys allows for the redistribution of oxygen in the rhenium-chromium metal alloys or rhenium alloys to minimize the tendency of micro-cracking in the rhenium-chromium metal alloys or rhenium alloys during the forming of the rhenium-chromium metal alloys or rhenium alloys at least partially into a medical device, and/or during the use and/or expansion of the medical device in a body. The atomic ratio of carbon to oxygen in the rhenium-chromium metal alloys or rhenium alloys is believed to facilitate in minimizing the tendency of micro-cracking in the rhenium-chromium metal alloys or rhenium alloys and improve the degree of elongation of the rhenium-chromium metal alloys or rhenium alloys, both of which can affect one or more physical properties of the rhenium-chromium metal alloys or rhenium alloys that are useful or desired in forming and/or using the medical device. The carbon to oxygen atomic ratio can be as low as about 0.2:1 (e.g., 0.2:1 to 50:1 and all values and ranges therebetween). In one non-limiting formulation, the carbon to oxygen atomic ratio in the rhenium-chromium metal alloys or rhenium alloys is generally at least about 0.3:1. Typically the carbon content of the rhenium-chromium metal alloy is less than about 0.1 wt. % (e.g., 0-0.0999999 wt. % and all values and ranges therebetween), and more typically 0-0.01 wt. %. Carbon contents that are too large can adversely affect the physical properties of the rhenium-chromium metal alloys or rhenium alloys. Generally, the oxygen content is to be maintained at very low level. In one non-limiting formulation, the oxygen content is less than about 0.1 wt. % of the rhenium-chromium metal alloys or rhenium alloys (e.g., 0-0.0999999 wt. % and all values and ranges therebetween), and typically 0-0.01 wt. %. It is believed that the rhenium-chromium metal alloys or rhenium alloys will have a very low tendency to form micro-cracks during the formation of the medical device and after the medical device has been inserted into a patient by closely controlling the carbon to oxygen ration when the oxygen content exceeds a certain amount in the rhenium-chromium metal alloys or rhenium alloys. In one non-limiting arrangement, the carbon to oxygen atomic ratio in the rhenium-chromium metal alloys or rhenium alloys is at least about 2.5:1 when the oxygen content is greater than about 100 ppm in the rhenium-chromium metal alloys or rhenium alloys.

In another and/or alternative non-limiting aspect of the present disclosure, the rhenium-chromium metal alloys or rhenium alloys as discussed above optionally include a controlled amount of nitrogen; however, this is not required. Large amounts of nitrogen in the rhenium-chromium metal alloys or rhenium alloys can adversely affect the ductility of the rhenium-chromium metal alloys or rhenium alloys. This can in turn adversely affect the elongation properties of the rhenium-chromium metal alloys or rhenium alloys. A too high nitrogen content in the rhenium-chromium metal alloys or rhenium alloys can begin to cause the ductility of the rhenium-chromium metal alloys or rhenium alloys to unacceptably decrease, thus adversely affect one or more physical properties of the rhenium-chromium metal alloys or rhenium alloys that are useful or desired in forming and/or using the medical device. In one non-limiting formulation, the rhenium-chromium metal alloys or rhenium alloys includes less than about 0.001 wt. % nitrogen (e.g., 0 wt. % to 0.0009999 wt. % and all values and ranges therebetween). It is believed that the nitrogen content should be less than the content of carbon or oxygen in the rhenium-chromium metal alloys or rhenium alloys. In one non-limiting formulation, the atomic ratio of carbon to nitrogen is at least about 1.5:1 (e.g., 1.5:1 to 400:1 and all values and ranges therebetween). In another non-limiting formulation, the atomic ratio of oxygen to nitrogen is at least about 1.2:1 (e.g., 1.2:1 to 150:1 and all value and ranges therebetween).

In another and/or alternative non-limiting aspect of the present disclosure, the rhenium-chromium metal alloys or rhenium alloys as discussed above that is used to form all or part of the medical device 1) is optionally not clad, metal sprayed, plated, and/or formed (e.g., cold worked, hot worked, etc.) onto another metal, or 2) optionally does not have another metal or metal alloy metal sprayed, plated, clad, and/or formed onto the rhenium-chromium metal alloys or rhenium alloys.

In another and/or alternative non-limiting aspect of the present disclosure, the medical device can optionally be formed from a tube or rod of the rhenium-chromium metal alloys or rhenium alloys, or be formed into a shape that is at least 80% of the final net shape of the medical device.

In another and/or alternative non-limiting aspect of the present disclosure, the rhenium-chromium metal alloys or rhenium alloys as discussed above have several physical properties that positively affect the medical device when the medical device is at least partially formed of the rhenium-chromium metal alloys or rhenium alloys of the present disclosure. In one non-limiting embodiment of the disclosure, the average Vickers hardness of the rhenium-chromium metal alloys or rhenium alloys of the present disclosure used to at least partially form the medical device is optionally at least about 150 Vickers (e.g., 150-300 Vickers and all values and ranges therebetween), and typically 160-240 Vickers; however, this is not required. The rhenium-chromium metal alloys or rhenium alloys of the present disclosure generally has an average hardness that is greater than stainless steel. In another and/or alternative non-limiting embodiment of the disclosure, the average ultimate tensile strength of the rhenium-chromium metal alloys or rhenium alloys of the present disclosure is optionally at least about 125 ksi (e.g., 125-300 ksi and all values and ranges therebetween); however, this is not required. In still another and/or alternative non-limiting embodiment of the disclosure, the average yield strength of the rhenium-chromium metal alloys or rhenium alloys of the present disclosure is optionally at least about 100 ksi (e.g., 100-275 ksi and all values and ranges therebetween); however, this is not required. In yet another and/or alternative non-limiting embodiment of the disclosure, the average grain size of the rhenium-chromium metal alloys or rhenium alloys of the present disclosure used to at least partially form the medical device is optionally no greater than about 4 ASTM (e.g., 4 ASTM to 20 ASTM using ASTM E112 and all values and ranges therebetween, e.g., 0.35 micron to 90 micron, and all values and ranges therebetween). The small grain size of the rhenium-chromium metal alloys or rhenium alloys of the present disclosure enables the medical device to have the desired elongation and ductility properties useful in enabling the medical device to be formed, crimped, and/or expanded.

In another and/or alternative non-limiting embodiment of the disclosure, the average tensile elongation of the rhenium-chromium metal alloys or rhenium alloys as discussed above that are used to at least partially form the medical device optionally have an average tensile strength of at least about 25% (e.g., 25-50% average tensile elongation and all values and ranges therebetween). An average tensile elongation of at least 25% for the rhenium-chromium metal alloys or rhenium alloys is useful to facilitate in the medical device being properly expanded when positioned in the treatment area of a body. A medical device that does not have an average tensile elongation of at least about 25% may be more prone to the formation of micro-cracks and/or break during the forming, crimping, and/or expansion of the medical device. The unique combination of the metals in the rhenium-chromium metal alloys or rhenium alloys of the present disclosure in combination with achieving the desired purity and composition of the alloy and the desired grain size of the rhenium-chromium metal alloys or rhenium alloys results in 1) a medical device having the desired high ductility at about room temperature, 2) a medical device having the desired amount of tensile elongation, 3) a homogeneous or solid solution of the rhenium-chromium metal alloys or rhenium alloys having high radiopacity, 4) a reduction or prevention of micro-crack formation and/or breaking of the rhenium-chromium metal alloys or rhenium alloys of the present disclosure tube when the tube is sized and/or cut to form the medical device, 5) a reduction or prevention of micro-crack formation and/or breaking of the medical device when the device is crimped, 6) a reduction or prevention of micro-crack formation and/or breaking of the medical device when the medical device is bent and/or expanded in a body, 7) a medical device having the desired ultimate tensile strength and yield strength, 8) a medical device having very thin wall thicknesses and still having the desired radial forces needed to retain the medical device on an open state when expanded, 9) a medical device that exhibits less recoil when the medical device is crimped onto a delivery system and/or expanded in a body, 10) a medical device that exhibits improved conformity to the shape of the treatment area in the body when the medical device is expanded in a body, 11) a medical device that exhibits improved fatigue ductility, and/or 12) a medical device that exhibits improved durability.

In another and/or alternative non-limiting aspect of the present disclosure, the rhenium-chromium metal alloys or rhenium alloys as discussed above are optionally at least partially formed by a swaging process; however, this is not required. In one non-limiting embodiment, swaging is performed on the rhenium-chromium metal alloys or rhenium alloys to at least partially or fully achieve final dimensions of one or more portions of the medical device. The swaging dies can be shaped to fit the final dimension of the medical device; however, this is not required. Where there are undercuts of hollow structures in the medical device (which is not required), a separate piece of metal can be placed in the undercut to at least partially fill the gap. The separate piece of metal (when used) can be designed to be later removed from the undercut; however, this is not required. The swaging operation can be performed on the medical device in the areas to be hardened. For a round or curved portion of a medical device, the swaging can be rotary. For non-round portion of the medical device, the swaging of the non-round portion of the medical device can be performed by non-rotating swaging dies. The dies can optionally be made to oscillate in radial and/or longitudinal directions instead of or in addition to rotating. The medical device can optionally be swaged in multiple directions in a single operation or in multiple operations to achieve a hardness in desired location and/or direction of the medical device. Swaging temperatures for a particular rhenium-chromium metal alloy can vary. For the rhenium-chromium metal alloys or rhenium alloys, the swaging temperature can be from room temperature (RT) (e.g., 10-27° C. and all values and ranges therebetween) to about 400° C. (e.g., 10-400° C. and all values and ranges therebetween) if the swaging is conducted in air or an oxidizing environment. The swaging temperature can be increased to up to about 1500° C. (e.g., 10-1550° C. and all values and ranges therebetween) if the swaging process is performed in a controlled neutral or non-reducing environment (e.g., inert environment). The rhenium-chromium metal alloys or rhenium alloys can be optionally preheated prior to being subjected to a swaging process. In one non-limiting arrangement, the rhenium-chromium metal alloys or rhenium alloys is preheated (e.g., 400-1550° C. and all values and ranges therebetween; 1350-1450° C.) for 1-200 minutes (and all values and ranges therebetween; 5-45 minutes). The swaging process can be conducted by repeatedly hammering the medical device at the location to be hardened at the desired swaging temperature. The rhenium-chromium metal alloys or rhenium alloys can optionally be first annealed to soften and then machined the rhenium-chromium metal alloys or rhenium alloys into a desired shape. After the rhenium-chromium metal alloys or rhenium alloys is shaped, the rhenium-chromium metal alloys or rhenium alloys can be re-hardened. The hardening of the rhenium-chromium metal alloys or rhenium alloys can improve the wear resistance and/or shape retention of the metal alloy. The rhenium-chromium metal alloys or rhenium alloys of the medical generally cannot be re-hardened by annealing, thus a special rehardening processes is required. Such rehardening can be achieved by the swaging process of the present disclosure. In one non-limiting embodiment, during the swaging process ions of boron and/or nitrogen are optionally allowed to impinge upon rhenium atoms in the rhenium-chromium metal alloys or rhenium alloys so as to form ReB₂, ReN₂ and/or ReN₃; however, this is not required. It has been found that ReB₂, ReN₂ and/or ReN₃ are ultra-hard compounds. In one non-limiting process, the metal for the medical device can be machined and shape into the medical device when the metal is in a less hardened state.

In another and/or alternative non-limiting aspect of the present disclosure, the rhenium-chromium metal alloys or rhenium alloys as discussed above can optionally be nitrided. The nitrided layer on the rhenium-chromium metal alloys or rhenium alloys can optionally function as a lubricating surface during the optional drawing of the rhenium-chromium metal alloys or rhenium alloys when partially or fully forming the medical device. After the rhenium-chromium metal alloys or rhenium alloys is nitrided, the rhenium-chromium metal alloys or rhenium alloys is typically cleaned; however, this is not required. During the nitriding process, the surface of the rhenium-chromium metal alloys or rhenium alloys is modified by the presence of nitrogen. The nitriding process can be by gas nitriding, salt bath nitriding, or plasma nitriding. The rhenium-chromium metal alloys or rhenium alloys can optionally be exposed to argon and/or hydrogen gas prior to the nitriding process to clean and/or preheat the rhenium-chromium metal alloys or rhenium alloys. These gases can be optionally used to clean oxide layers and/or solvents from the surface of the rhenium-chromium metal alloys or rhenium alloys. During the nitriding process, the rhenium-chromium metal alloys or rhenium alloys can optionally be exposed to hydrogen gas to inhibit or prevent the formation of oxides on the surface of the rhenium-chromium metal alloys or rhenium alloys. The thickness of the nitrided surface layer is less than about 1 mm. In one non-limiting embodiment, the thickness of the nitride surface layer is at least about 50 nanometer and less than about 1 mm (and all values and ranges therebetween). In another non-limiting embodiment, the thickness of the nitrided surface layer is at least about 50 nanometer and less than about 0.1 mm. Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the rhenium-chromium metal alloys or rhenium alloys, and typically less than each of the two primary components of the rhenium-chromium metal alloys or rhenium alloys. For example, when the rhenium-chromium metal alloys or rhenium alloys is nitrided, the weight percent of the nitrogen in the nitrided surface layer is less than a weight percent of the rhenium in the nitrided surface layer. As can be appreciated, the complete outer surface of the rhenium-chromium metal alloys or rhenium alloys can be nitrided or a portion of the outer surface of the rhenium-chromium metal alloys or rhenium alloys can be nitrided. Nitriding only selected portions of the outer surface of the rhenium-chromium metal alloys or rhenium alloys can be used to obtain different surface characteristics of the rhenium-chromium metal alloys or rhenium alloys; however, this is not required. As can be appreciated, the final formed rhenium-chromium metal alloy can include a nitrided outer surface. The nitriding process for the rhenium-chromium metal alloys or rhenium alloys can be used to increase surface hardness and/or wear resistance of the medical device, and/or to inhibit or prevent discoloration of the rhenium-chromium metal alloys or rhenium alloys (e.g., discoloration by oxidation, etc.). For example, the nitriding process can be used to increase the wear resistance of articulation surface or surfaces wear on the rhenium-chromium metal alloys or rhenium alloys used in the medical device to extend the life of the medical device, and/or increase the wear life of mating surfaces on the medical device, and/or to reduce particulate generation from use of the medical device, and/or to maintain the outer surface appearance of the rhenium-chromium metal alloys or rhenium alloys on the medical device.

In another and/or alternative non-limiting aspect of the present disclosure, the rhenium-chromium metal alloy, just prior to or after being partially or fully formed into the desired medical device, can optionally be cleaned, polished, sterilized, nitrided, etc., for final processing of the rhenium-chromium metal alloy.

In another and/or alternative non-limiting aspect of the present disclosure, the use of the rhenium-chromium metal alloys or rhenium alloys as discussed above to partially or fully form the medical device can be used to increase the strength and/or hardness and/or durability of the medical device as compared with stainless steel or chromium-cobalt alloys or titanium alloys; thus, less quantity of rhenium-chromium metal alloy can be used in the medical device to achieve similar strengths as compared to medical devices formed of different metals. As such, the resulting medical device can be made smaller and less bulky by use of the rhenium-chromium metal alloys or rhenium alloys without sacrificing the strength and durability of the medical device. Such a medical device can have a smaller profile, thus can be inserted in smaller areas, openings, and/or passageways. The rhenium-chromium metal alloys or rhenium alloys also can increase the radial strength of the medical device. For example, the thickness of the walls of the medical device and/or the wires used to at least partially form the medical device can be made thinner and achieve a similar or improved radial strength as compared with thicker-walled medical devices formed of stainless steel, titanium alloys, or cobalt and chromium alloys. The rhenium-chromium metal alloys or rhenium alloys also can improve stress-strain properties, bendability and flexibility of the medical device, thus increase the life of the medical device. For instance, the medical device can be used in regions that subject the medical device to bending. Due to the improved physical properties of the medical device from the rhenium-chromium metal alloys or rhenium alloys, the medical device has improved resistance to fracturing in such frequent bending environments. In addition or alternatively, the improved bendability and flexibility of the medical device due to the use of the rhenium-chromium metal alloys or rhenium alloys can enable the medical device to be more easily inserted into various regions of a body. The rhenium-chromium metal alloys or rhenium alloys can also reduce the degree of recoil during the crimping and/or expansion of the medical device. For example, the medical device better maintains its crimped form and/or better maintains its expanded form after expansion due to the use of the rhenium-chromium metal alloys or rhenium alloys. As such, when the medical device is to be mounted onto a delivery device when the medical device is crimped, the medical device better maintains its smaller profile during the insertion of the medical device into various regions of a body. Also, the medical device better maintains its expanded profile after expansion to facilitate in the success of the medical device in the treatment area. In addition to the improved physical properties of the medical device by use of the rhenium-chromium metal alloys or rhenium alloys, the rhenium-chromium metal alloys or rhenium alloys has improved radiopaque properties as compared to standard materials such as stainless steel or cobalt-chromium alloy, thus reducing or eliminating the need for using marker materials on the medical device.

In another and/or alternative non-limiting aspect of the present disclosure, the medical device can optionally contain and/or be coated with one or more agents that facilitate in the success of the medical device and/or treated area. The term “agent” includes, but is not limited to, a substance, pharmaceutical, biologic, veterinary product, drug, and analogs or derivatives otherwise formulated and/or designed to prevent, inhibit, and/or treat one or more clinical and/or biological events, and/or to promote healing. Non-limiting examples of clinical events that can be addressed by one or more agents include, but are not limited to, viral, fungus and/or bacterial infection; vascular diseases and/or disorders, digestive diseases and/or disorders, reproductive diseases and/or disorders, lymphatic diseases and/or disorders, cancer, implant rejection, pain, nausea, swelling, arthritis, bone diseases and/or disorders, organ failure, immunity diseases and/or disorders, cholesterol problems, blood diseases and/or disorders, lung diseases and/or disorders, heart diseases and/or disorders, brain diseases and/or disorders, neuralgia diseases and/or disorders, kidney diseases and/or disorders, ulcers, liver diseases and/or disorders, intestinal diseases and/or disorders, gallbladder diseases and/or disorders, pancreatic diseases and/or disorders, psychological disorders, respiratory diseases and/or disorders, gland diseases and/or disorders, skin diseases and/or disorders, hearing diseases and/or disorders, oral diseases and/or disorders, nasal diseases and/or disorders, eye diseases and/or disorders, fatigue, genetic diseases and/or disorders, burns, scarring and/or scars, trauma, weight diseases and/or disorders, addiction diseases and/or disorders, hair loss, cramp, muscle spasms, tissue repair, nerve repair, neural regeneration, and/or the like. The type and/or amount of agent included in the medical device and/or coated on medical device can vary. When two or more agents are included in and/or coated on the medical device, the amount of the two or more agents can be the same or different. The one or more agents can be coated on and/or impregnated in the medical device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the disclosure, the type and/or amount of agent included on, in, and/or in conjunction with the medical device is generally selected for the treatment of one or more medical treatments. The amount of two or more agents on, in, and/or used in conjunction with the medical device can be the same or different. The one or more agents, when used on and/or in the medical device, can optionally be released in a controlled manner so the area in question to be treated is provided with the desired dosage of agent over a sustained period of time. As can be appreciated, controlled release of one or more agents on the medical device is not always required and/or desirable. As such, one or more of the agents on and/or in the medical device can be uncontrollably released from the medical device during and/or after insertion of the medical device in the treatment area. It can also be appreciated that one or more agents on and/or in the medical device can be controllably released from the medical device and one or more agents on and/or in the medical device can be uncontrollably released from the medical device. It can also be appreciated that one or more agents on and/or in one region of the medical device can be controllably released from the medical device and one or more agents on and/or in the medical device can be uncontrollably released from another region on the medical device. As such, the medical device can be designed such that 1) all the agent on and/or in the medical device is controllably released, 2) some of the agent on and/or in the medical device is controllably released and some of the agent on the medical device is non-controllably released, or 3) none of the agent on and/or in the medical device is controllably released. The medical device can also be designed such that the rate of release of the one or more agents from the medical device is the same or different. The medical device can also be designed such that the rate of release of the one or more agents from one or more regions on the medical device is the same or different. Non-limiting arrangements that can be used to control the release of one or more agents from the medical device include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the medical device and at least partially coat or cover such pores, passageway, cavities, etc., with one or more polymers. As can be appreciated, other or additional arrangements can be used to control the release of one or more agents from the medical device. The one or more polymers, when used to at least partially control the release of one or more agents from the medical device, can be porous or non-porous. The one or more agents can be inserted into and/or applied to one or more surface structures and/or micro-structures on the medical device, and/or be used to at least partially form one or more surface structures and/or micro-structures on the medical device. As such, the one or more agents on the medical device can be 1) coated on one or more surface regions of the medical device, 2) inserted and/or impregnated in one or more surface structures and/or micro-structures, etc., of the medical device, and/or 3) form at least a portion or be included in at least a portion of the structure of the medical device. When the one or more agents are coated on the medical device, the one or more agents can 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials. As can be appreciated, many other coating arrangements can be additionally or alternatively used. When the one or more agents are optionally inserted and/or impregnated in one or more internal structures, surface structures and/or micro-structures of the medical device, 1) one or more other coating materials can be applied at least partially over the one or more internal structures, surface structures, and/or micro-structures of the medical device, and/or 2) one or more polymers can be combined with one or more agents. As such, the one or more agents can be 1) embedded in the structure of the medical device, 2) positioned in one or more internal structures of the medical device, 3) encapsulated between two polymer coatings, 4) encapsulated between the base structure and a polymer coating, 5) mixed in the base structure of the medical device that includes at least one polymer coating, or 6) one or more combinations of 1, 2, 3, 4, and/or 5. In addition or alternatively, the one or more coating of the one or more polymers on the medical device can include 1) one or more coatings of non-porous polymers, 2) one or more coatings of a combination of one or more porous polymers and one or more non-porous polymers, 3) one or more coating of porous polymer, or 4) one or more combinations of options 1, 2, and 3.

In another and/or alternative non-limiting aspect of the present disclosure, different agents can optionally be located in and/or between different polymer coating layers and/or on the structure of the medical device. As can also be appreciated, many other and/or additional coating combinations and/or configurations can be used. The concentration of one or more agents, the type of polymer, the type and/or shape of internal structures in the medical device, and/or the coating thickness of one or more agents can be used to control the release time, the release rate, and/or the dosage amount of one or more agents; however, other or additional combinations can be used. As such, the agent and polymer system combination and location on the medical device can be numerous. As can also be appreciated, one or more agents can be deposited on the top surface of the medical device to provide an initial uncontrolled burst effect of the one or more agents prior to the 1) controlled release of the one or more agents through one or more layers of a polymer system that include one or more non-porous polymers, and/or 2) uncontrolled release of the one or more agents through one or more layers of a polymer system. The one or more agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

In another and/or alternative non-limiting aspect of the present disclosure, a variety of polymers can optionally be coated on the medical device and/or be used to form at least a portion of the medical device. The one or more polymers can be used on the medical device for a variety of reasons such as, but not limited to, 1) forming a portion of the medical device, 2) improving a physical property of the medical device (e.g., improve strength, improve durability, improve biocompatibility, reduce friction, etc.), 3) forming a protective coating on one or more surface structures on the medical device, 4) at least partially forming one or more surface structures on the medical device, and/or 5) at least partially controlling a release rate of one or more agents from the medical device. As can be appreciated, the one or more polymers can have other or additional uses on the medical device. The one or more polymers can be porous, non-porous, biostable, biodegradable (i.e., dissolves, degrades, is absorbed, or any combination thereof in the body), and/or biocompatible. When the medical device is coated with one or more polymers, the polymer can include 1) one or more coatings of non-porous polymers, 2) one or more coatings of a combination of one or more porous polymers and one or more non-porous polymers, 3) one or more coatings of one or more porous polymers and one or more coatings of one or more non-porous polymers, 4) one or more coating of porous polymer, or 5) one or more combinations of options 1, 2, 3, and 4. The thickness of one or more of the polymer layers can be the same or different. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing, and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification. The thickness of each polymer layer is generally at least about 0.01 μm and is generally less than about 150 μm (e.g., 0.01 μm to 150 μm and all values and ranges therebetween); however, other thicknesses can be used. In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In another and/or alternative non-limiting aspect of the present disclosure, the medical device, when including and/or is coated with one or more agents, can include and/or can be coated with one or more agents that are the same or different in different regions of the medical device and/or have differing amounts and/or concentrations in differing regions of the medical device. For instance, the medical device can 1) be coated with and/or include one or more biologicals on at least one portion of the medical device and at least another portion of the medical device is not coated with and/or includes agent; 2) be coated with and/or include one or more biologicals on at least one portion of the medical device that is different from one or more biologicals on at least another portion of the medical device; and/or 3) be coated with and/or include one or more biologicals at a concentration on at least one portion of the medical device that is different from the concentration of one or more biologicals on at least another portion of the medical device.

In another and/or alternative non-limiting aspect of the present disclosure, one or more portions of the medical device can optionally 1) include the same or different agents, 2) include the same or different amount of one or more agents, 3) include the same or different polymer coatings, 4) include the same or different coating thicknesses of one or more polymer coatings, 5) have one or more portions of the medical device controllably release and/or uncontrollably release one or more agents, and/or 6) have one or more portions of the medical device controllably release one or more agents and one or more portions of the medical device uncontrollably release one or more agents.

In another and/or alternative non-limiting aspect of the present disclosure, one or more surfaces of the medical device can optionally be treated to achieve the desired coating properties of the one or more agents and one or more polymers coated on the medical device. Such surface treatment techniques include, but are not limited to, cleaning, buffing, smoothing, nitriding, annealing, swaging, rolling, cold working, etching (chemical etching, plasma etching, etc.), etc. As can be appreciated, other or additional surface treatment processes can be used prior to the coating of one or more agents and/or polymers on the surface of the medical device.

In another and/or alternative non-limiting aspect of the present disclosure, the medical device can optionally include a marker material that facilitates enabling the medical device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). The marker material can form all or a portion of the medical device and/or be coated on one or more portions (flaring portion and/or body portion, at ends of medical device, at or near transition of body portion and flaring section, etc.) of the medical device. The location of the marker material can be on one or multiple locations on the medical device. The size of the one or more regions including the marker material can be the same or different. The marker material can be spaced at defined distances from one another to form ruler-like markings on the medical device to facilitate in the positioning of the medical device in a body passageway. The marker material can be a rigid or flexible material. The marker material can be a biostable or biodegradable material.

In another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, micro-molding, etc.); however, other or additional manufacturing techniques can be used.

In another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology.

In another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm. As can be appreciated, when the medical device includes one or more surface structures, 1) all the surface structures can be micro-structures, 2) all the surface structures can be non-micro-structures, or 3) a portion of the surface structures can be micro-structures and a portion can be non-micro-structures. Typically, the micro-structures (when formed) extend from or into the outer surface no more than about 400 microns (0.01-400 microns and all values and ranges therebetween), and more typically less than about 300 microns, and more typically about 15-250 microns; however, other sizes can be used. The micro-structures can be clustered together or disbursed throughout the surface of the medical device. Similar shaped and/or sized micro-structures and/or surface structures can be used, or different shaped and/or sized micro-structures can be used. When one or more surface structures and/or micro-structures are designed to extend from the surface of the medical device, the one or more surface structures and/or micro-structures can be formed in the extended position and/or be designed to extend from the medical device during and/or after deployment of the medical device in a treatment area. The micro-structures and/or surface structures can be designed to contain and/or be fluidly connected to a passageway, cavity, etc.; however, this is not required. The one or more surface structures and/or micro-structures can be used to engage and/or penetrate surrounding tissue or organs once the medical device has been positioned on and/or in a patient; however, this is not required. The one or more surface structures and/or micro-structures can be used to facilitate in forming and maintaining a shape of a medical device. In one non-limiting embodiment, the one or more surface structures and/or micro-structures can be at least partially formed of an agent and/or be formed of a polymer. One or more of the surface structures and/or micro-structures can include one or more internal passageways that can include one or more materials (e.g., agent, polymer, etc.); however, this is not required. The one or more coatings and/or one or more surface structures and/or micro-structures of the medical device can be used for a variety of purposes such as, but not limited to, 1) increasing the bonding and/or adhesion of one or more agents, adhesives, marker materials, and/or polymers to the medical device, 2) changing the appearance or surface characteristics of the medical device, and/or 3) controlling the release rate of one or more agents. The one or more micro-structures and/or surface structures can be biostable, biodegradable, etc. The medical device or one or more regions of the medical device can be at least partially covered and/or filled with a protective material to at least partially protect one or more regions of the medical device, and/or one or more micro-structures, and/or surface structures on the medical device from damage. The protective material can include one or more polymers previously identified above. The protective material can be 1) biostable and/or biodegradable and/or 2) porous and/or non-porous.

In another and/or alternative aspect of the disclosure, the medical device can optionally be an expandable device that can be expanded by use of some other device (e.g., balloon, etc.). The expandable medical device can be fabricated from a material that has no or substantially no shape-memory characteristics.

In another and/or alternative non-limiting aspect of the present disclosure, there is optionally provided a near net process for a frame or other metal component of the medical device.

In another and/or alternative non-limiting aspect of the present disclosure, the rhenium-chromium metal alloys or rhenium alloys as discussed above can be formed into a rod, tube, green part, near net part, etc. by a sintering process, a cold isostatic pressing (CIP) process and/or a hot isostatic pressing (HIP) process. The particle size of the metal particles that are used in the sintering process, the cold isostatic pressing (CIP) process and/or the hot isostatic pressing (HIP) process are generally less than about 100 mesh (e.g., less than 150 microns; 2-80 microns; 5-40 microns). The metal particles can be high pure metal particles formed of a single element, and/or can be highly pure metal particles formed of two of more elements. The purity of the metal particles should be selected so that the metal particles contain very low levels elemental impurities (e.g., 99+% purity; 99.5+%; purity 99.99+% purity). In one non-limiting embodiment, the rod, the tube, the green part, the near net part, etc. is formed by a sintering process. The sintering process occurs at a temperature of at least 1600° C. (e.g., 1600-2600° C. and all values and ranges therebetween). The sintering process can optionally occur in a non-oxidizing environment (e.g., hydrogen atmosphere, etc.). The sintering process can optionally occur under a vacuum or under pressures exceeding 1 atm. (0.0074-300 tsi and all values and ranges therebetween). In one non-limiting example, the rod, the tube, the green part, the near net part, etc. is at least partially formed of the rhenium-chromium metal alloys or rhenium alloys by a sintering process wherein the sintering temperature is about 1550° C.-1850° C. (and all values and ranges therebetween) for about 2-40 hours (and all values and ranges therebetween) in a non-oxidizing environment. In another non-limiting embodiment, a rod, tube, green part, near net part, etc. is at least partially formed of the rhenium-chromium metal alloys or rhenium alloys by a hot isostatic pressing (HIP) process. In one non-limiting arrangement, the hot isostatic pressing (HIP) process optionally occurs at a temperature of 1700° C.-2100° C. (and all values and ranges therebetween) for about 1-6 hours (and all values and ranges therebetween). The hot isostatic pressing (HIP) process can optionally occur in a non-oxidizing environment (e.g., hydrogen atmosphere, etc.). The hot isostatic pressing (HIP) process occurs under a vacuum or under pressures exceeding 1 atm. (0.0074-300 tsi and all values and ranges therebetween).

In another and/or alternative non-limiting aspect of the present disclosure, the formed rod, tube, green part, near net part, etc. of the rhenium-chromium metal alloys or rhenium alloys as discussed above can be optionally strengthen by subjecting the rhenium-chromium metal alloys or rhenium alloys to cold working. In one non-limiting embodiment, the rod, tube, green part, near net part, etc. is at least partially formed by pressed together the metal particles and then sintering the press together metal particles. Thereafter, the sintered part is can optionally be again pressed to increase its mechanical strength by imparting cold work into the sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered and re-pressed) meets the dimensional requirements of the final formed part. For the rhenium-chromium metal alloys or rhenium alloys or rhenium alloys, a post-sintered pressure of 1-300 tsi (1 ton per square inch) (and all values and ranges therebetween) can be used.

In still yet another and/or alternative non-limiting aspect of the present disclosure, there is optionally provided a press of near net or finished part composite. The process of pressing metals into near net of finished parts is well established; however, pressing a composite structure formed of metal powder and polymer for purposes of making complex part geometries and foam-like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation, on the metal substrate, and the polymer residual has some desired biological affect (e.g., masking the metal from the body by encapsulation, promotion of cellular attachment and growth). The polymer and metal powders can be of varying sizes to create multiple voids—some large to create a pathway for cellular growth, and some small to create a ruff surface to promote cellular attachment.

In a further and/or alternative non-limiting aspect of the present disclosure, the rhenium-chromium metal alloys or rhenium alloys as discussed above that is used to at least partially form the medical device is initially formed into a blank, a rod, a tube, etc., and then finished into final form by one or more finishing processes. The rhenium-chromium metal alloys or rhenium alloys blank, rod, tube, etc., can be formed by various techniques such as, but not limited to, 1) melting the rhenium-chromium metal alloys or rhenium alloys and/or metals that form the rhenium-chromium metal alloys or rhenium alloys (e.g., vacuum arc melting, etc.) and then extruding and/or casting the rhenium-chromium metal alloys or rhenium alloys into a blank, rod, tube, etc., 2) melting the rhenium-chromium metal alloys or rhenium alloys and/or metals that form the rhenium-chromium metal alloys or rhenium alloys, forming a metal strip, and then rolling and welding the strip into a blank, rod, tube, etc., or 3) consolidating the metal powder of the rhenium-chromium metal alloys or rhenium alloys and/or metal powder of metals that form the rhenium-chromium metal alloys or rhenium alloys into a blank, rod, tube, etc. When the rhenium-chromium metal alloys or rhenium alloys is formed into a blank, the shape and size of the blank is non-limiting. In one non-limiting process, the near net medical device, blank, rod, tube, etc., can be formed from one or more ingots of metal or rhenium-chromium metal alloy. In one non-limiting process, an arc melting process (e.g., vacuum arc melting process, etc.) can be used to form the near net medical device, blank, rod, tube, etc. In another non-limiting process, rhenium powder and tungsten powder and optionally molybdenum powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device, blank, rod, tube, etc. It can be appreciated that other or additional processes can be used to form the rhenium-chromium metal alloys or rhenium alloys.

In still yet a further and/or alternative non-limiting aspect of the present disclosure, the near net medical device, blank, rod, tube, etc., can be resized to the desired dimension of the medical device. In one non-limiting embodiment, the cross-sectional area or diameter of the near net medical device, blank, rod, tube, etc., is reduced to a final near net medical device, blank, rod, tube, etc. dimension in a single step or by a series of steps. The reduction of the outer cross-sectional area or diameter of the near net medical device, blank, rod, tube, etc., may be obtained by centerless grinding, turning, electropolishing, drawing process, grinding, laser cutting, shaving, polishing, EDM cutting, etc. The outer cross-sectional area or diameter size of the near net medical device, blank, rod, tube, etc., can be reduced by the use of one or more drawing processes; however, this is not required. During the drawing process, care should be taken to not form micro-cracks in the near net medical device, blank, rod, tube, etc., during the reduction of the near net medical device, blank, rod, tube, etc., outer cross-sectional area or diameter.

The use of the rhenium-chromium metal alloys or rhenium alloys as discussed above to form all or a portion of the medical device can result in several advantages over medical devices formed from other materials. These advantages include, but are not limited to:

The rhenium-chromium metal alloys or rhenium alloys has increased strength and/or hardness compared with stainless steel or chromium-cobalt alloys or titanium alloys, thus a lesser quantity of rhenium-chromium metal alloy can be used in the medical device to achieve similar strengths compared to medical devices formed of different metals. As such, the resulting medical device can be made smaller and less bulky by use of the rhenium-chromium metal alloys or rhenium alloys without sacrificing the strength and durability of the medical device. The medical device can also have a smaller profile, thus can be inserted into smaller areas, openings, and/or passageways. The thinner struts of rhenium-chromium metal alloy to form the frame or other portions of the medical device can be used to form a frame or other portion of the medical device having a strength that would require thicker struts or other structures of the medical device when formed by stainless steel, chromium-cobalt alloys, or titanium alloys.

The increased strength of the rhenium-chromium metal alloys or rhenium alloys also results in the increased radial strength of the medical device. For instance, the thickness of the walls of the medical device can be made thinner and achieve a similar or improved radial strength as compared with thicker-walled medical devices formed of stainless steel, cobalt and chromium alloy, or titanium alloy.

The rhenium-chromium metal alloys or rhenium alloys has improved stress-strain properties, bendability properties, elongation properties, and/or flexibility properties of the medical device compared with stainless steel or chromium-cobalt alloys, thus resulting in an increased life for the medical device. For instance, the medical device can be used in regions that subject the medical device to repeated bending. Due to the improved physical properties of the medical device from the rhenium-chromium metal alloys or rhenium alloys, the medical device has improved resistance to fracturing in such frequent bending environments. These improved physical properties at least in part result from the composition of the rhenium-chromium metal alloys or rhenium alloys, the grain size of the rhenium-chromium metal alloys or rhenium alloys, the carbon, oxygen, and nitrogen content of the rhenium-chromium metal alloys or rhenium alloys, and/or the carbon/oxygen ratio of the rhenium-chromium metal alloys or rhenium alloys.

The rhenium-chromium metal alloys or rhenium alloys has a reduced degree of recoil during the crimping and/or expansion of the medical device compared with stainless steel or chromium-cobalt alloys or titanium alloys. The medical device formed of the rhenium-chromium metal alloys or rhenium alloys better maintains its crimped form and/or better maintains its expanded form after expansion due to the use of the rhenium-chromium metal alloys or rhenium alloys. As such, when the medical device is to be mounted onto a delivery device when the medical device is crimped, the medical device better maintains its smaller profile during the insertion of the medical device in a body passageway. Also, the medical device better maintains its expanded profile after expansion to facilitate in the success of the medical device in the treatment area.

The use of rhenium-chromium metal alloy in the medical device medical device results in the medical device better conforming to an irregularly shaped body passageway when expanded in the body passageway as compared to a medical device formed by stainless steel, chromium-cobalt alloys, or titanium alloys.

The rhenium-chromium metal alloys or rhenium alloys has improved radiopaque properties compared to standard materials such as stainless steel or cobalt-chromium alloy, thus reducing or eliminating the need for using marker materials on the medical device. For instance, the rhenium-chromium metal alloys or rhenium alloys is at least about 10-20% more radiopaque than stainless steel or cobalt-chromium alloy.

The rhenium-chromium metal alloys or rhenium alloys has improved fatigue ductility when subjected to cold-working as compared to the cold-working of stainless steel, chromium-cobalt alloys, or titanium alloys.

The rhenium-chromium metal alloys or rhenium alloys has improved durability compared to stainless steel, chromium-cobalt alloys, or titanium alloys.

The rhenium-chromium metal alloys or rhenium alloys has improved hydrophilicity compared to stainless steel, chromium-cobalt alloys, or titanium alloys.

The rhenium-chromium metal alloys or rhenium alloys has reduced ion release in the body passageway as compared to stainless steel, chromium-cobalt alloys, or titanium alloys.

The rhenium-chromium metal alloys or rhenium alloys is less of an irritant to the body than stainless steel, cobalt-chromium alloy, or titanium alloys, thus can result in reduced inflammation, faster healing, and increased success rates of the medical device. When the medical device is expanded in a body passageway, some minor damage to the interior of the passageway can occur. When the body begins to heal such minor damage, the body has less adverse reaction to the presence of the rhenium-chromium metal alloys or rhenium alloys compared to other metals such as stainless steel, cobalt-chromium alloy, or titanium alloy.

The medical devices which include expandable metal frames that are at least partially formed of the rhenium-chromium metal alloys or rhenium alloys exhibit reduced amount of recoil, improved bending conformity, and greater radial strength compared to expandable frames formed of stainless steel, cobalt-chromium alloy, and TiAlV alloy, thereby resulting in the following non-limiting advantages compared to expandable frames formed of stainless steel, cobalt-chromium alloy, or TiAlV alloy: 1) the formation of a frame for a medical device having thinner posts, struts, and/or strut joints which results in i) safer vascular access when inserting the medical device through a body passageway and to the treatment area, and/or ii) decreased risk of bleeding and/or damage to the body passageway and/or the treatment area when the medical device is delivered to the treatment area and/or expanded at the treatment area; 2) easier deliverability of the medical device to the treatment area which can result in i) decreased trauma to the body passageway (e.g., blood vessel, aortic arch trauma, etc.) during the insertion and/or expansion of the medical device at the treatment area, and/or ii) decreased risk of neuro complications-stroke; 3) less recoil which results in i) reduced crimping profile size, ii) increased conformability of the expanded medical device at the treatment area after expansion in the treatment area, iii) increased radial strength of the frame of the medical device after expansion at the treatment area, iv) only require a single crimping cycle to crimp the medical device on a balloon catheter or other type of delivery device, v) reduced incidence of damage to components of the medical device (e.g., struts, posts, strut joints, and/or other components of the expandable frame, leaflets, skirts, coatings, etc.) during the crimping, expansion, and operation of the medical device, vi) greater effective orifice area (EOA) of the medical device after expansion of the medical device, vi) decreased pulmonary valve regurgitation (PVR) after expansion of the medical device in the treatment area, and/or vii) require only a single expansion cycle of the balloon on the balloon catheter or other expansion mechanism to fully expand the medical device; and/or 4) creating a medical device having superior material biologic properties to i) improve tissue adhesion and/or growth on or about medical device, ii) reduce adverse tissue reactions with the medical device, iii) reduce toxicity of medical device, iv) potentially decrease in-valve thrombosis during the life of the medical device, and/or v) reduce the incidence of infection during the life of the medical device.

Medical devices, such as expandable medical devices (e.g., expandable heart valves, stents, etc.) that include the rhenium-chromium metal alloys or rhenium alloys in accordance with the present disclosure overcome several unmet needs that exist in expandable medical device that are formed of cobalt-chromium alloys, TiAlV alloys, and stainless steel. Such unmet needs addressed by the medical devices in accordance with the present disclosure include 1) not having to form a large hole in large arterial vessels or other blood vessels for initial insertion of the crimped medical device into the atrial vessel or other blood vessel, thereby reducing the incidence of lethal bleeding during a treatment; 2) enabling the medical device to be delivered and implanted in abnormally shaped heart valves or through an abnormally shaped arterial vessel due to calcination in the heart valve and/or calcination and/or plaque in the arterial vessel by creating a medical device (e.g., stent, prosthetic heart valve, etc.) that has a reduced crimped profile smaller than medical devices formed of cobalt-chromium alloys, TiAlV alloys, and stainless steel; 3) reducing the incidence of a perivalvular leak and/or other types of leakage about the implanted medical device when the medical device is expanded in the treatment region by using a frame formed of the rhenium-chromium metal alloys or rhenium alloys that better conforms to the shape of the abnormally shaped heart valve orifice upon expansion of the prosthetic heart valve as comparted to prior art prosthetic heart valves formed of cobalt-chromium alloys, TiAlV alloys, and stainless steel, thereby reducing the incidence of stroke and/or by increasing the incidence of success of the implanted medical device; 4) improving the radial strength of the expanded struts, posts, and/or strut joints in the expandable frame and the strength of the expandable frame itself after expansion the medical device; 5) reducing the amount of recoil of the expandable frame during the crimping and/or expansion of the expandable frame of the medical device; 6) enabling the medical device to be used in a heart that has a permanent pacemaker; 7) reducing the incidence of minor stroke during the insertion and operation of the medical device at the treatment area; 8) reducing the incidence of coronary ostium compromise; 9) improving foreshortening; 10) reducing further aortic valve calcification and/or calcification in a blood vessel after implantation of the medical device; 11) reducing the need for multiple crimping cycles when inserting the medical device on a catheter or other type of delivery system; 12) reducing the incidence of frame/stent fracture during the crimping and/or expansion of the medical device; 13) reducing the incidence of biofilm-endocarditis after implantation of the medical device; 14) reducing allergic reactions to the medical device after implantation of the medical device; 15) improving the hydrophilicity of the medical device to improve tissue growth on and/or about the implanted medical device, 16) reducing the magnetic susceptibility of the medical device, 17) reducing the toxicity of the medical device, 18) reducing the amount of metal ion release from the medical device, and/or 19) increasing the longevity of leaflets and/or stent/frame and/or other components of the medical device after insertion of the medical device.

One non-limiting object of the present disclosure is the provision of rhenium-chromium metal alloy in accordance with the present disclosure that can be used to partially or fully form a medical device.

Another and/or alternative non-limiting object of the present disclosure is the provision of a medical device that is partially or fully formed of the rhenium-chromium metal alloys or rhenium alloys of the present disclosure and which medical device has improved procedural success rates.

Another and/or alternative non-limiting object of the present disclosure is the provision of a method and process for forming the rhenium-chromium metal alloys or rhenium alloys in accordance with the present disclosure that inhibits or prevents the formation of micro-cracks during the processing of the rhenium-chromium metal alloys or rhenium alloys.

Another and/or alternative non-limiting object of the present disclosure is the provision of a medical device that is partially or fully formed of the rhenium-chromium metal alloys or rhenium alloys in accordance with the present disclosure and wherein the medical device has improved physical properties.

Another and/or alternative non-limiting object of the present disclosure is the provision of a medical device that is at least partially formed of the rhenium-chromium metal alloys or rhenium alloys in accordance with the present disclosure wherein the medical device has increased strength and/or hardness.

Another and/or alternative non-limiting object of the present disclosure is the provision of a medical device that at least partially includes the rhenium-chromium metal alloys or rhenium alloys in accordance with the present disclosure and which rhenium-chromium metal alloy enables the medical device to be formed with less material without sacrificing the strength of the medical device as compared to prior medical devices.

Another and/or alternative non-limiting object of the present disclosure is the provision of a method and process for forming the rhenium-chromium metal alloys or rhenium alloys in accordance with the present disclosure to inhibit or prevent the formation of micro-cracks during the processing of the rhenium-chromium metal alloys or rhenium alloys into a medical device.

Another and/or alternative non-limiting object of the present disclosure is the provision of a method and process for forming the rhenium-chromium metal alloys or rhenium alloys in accordance with the present disclosure that inhibits or prevents crack propagation and/or fatigue failure of the rhenium-chromium metal alloys or rhenium alloys.

Another and/or alternative non-limiting object of the present disclosure is the provision of a medical device that includes the rhenium-chromium metal alloys or rhenium alloys that has had a nitriding process to form a nitrided layer on the outer surface of the rhenium-chromium metal alloys or rhenium alloys.

Another and/or alternative non-limiting object of the present disclosure is the provision of a medical device that includes the rhenium-chromium metal alloys or rhenium alloys wherein the refractor alloy has been subjected to a swaging process.

Another and/or alternative non-limiting object of the present disclosure is the provision of a medical device that includes the rhenium-chromium metal alloys or rhenium alloys wherein the refractor alloy has been subjected to a cold-working process.

Another and/or alternative non-limiting object of the present disclosure is the provision of a medical device that includes the rhenium-chromium metal alloys or rhenium alloys that has increased strength and/or hardness compared with stainless steel, chromium-cobalt alloys, or titanium alloys.

Another and/or alternative non-limiting object of the present disclosure is the provision of a medical device that includes the rhenium-chromium metal alloys or rhenium alloys, thereby requiring a lesser quantity of rhenium-chromium metal alloy to achieve similar strengths compared to medical devices formed of different metals.

Another and/or alternative non-limiting object of the present disclosure is the provision of a medical device that includes the rhenium-chromium metal alloys or rhenium alloys wherein the medical device has a smaller crimped profile compared to medical devices formed of different metals.

Another and/or alternative non-limiting object of the present disclosure is the provision of a medical device that includes the rhenium-chromium metal alloys or rhenium alloys wherein the medical device has thinner walls and still achieves a similar or improved radial strength compared with thicker walled medical devices formed of stainless steel, chromium-cobalt alloy, or titanium alloys.

Another and/or alternative non-limiting object of the present disclosure is the provision of a medical device that includes the rhenium-chromium metal alloys or rhenium alloys wherein the medical device has improved stress-strain properties, bendability properties, elongation properties, and/or flexibility properties compared to medical devices formed of stainless steel, titanium steel, or chromium-cobalt alloys.

Another and/or alternative non-limiting object of the present disclosure is the provision of a medical device that includes the rhenium-chromium metal alloys or rhenium alloys wherein the medical device has an increased life compared to medical devices formed of stainless steel, titanium steel, or chromium-cobalt alloys.

Another and/or alternative non-limiting object of the present disclosure is the provision of a medical device that includes the rhenium-chromium metal alloys or rhenium alloys wherein the medical device has a reduced degree of recoil during the crimping and/or expansion of the medical device compared with stainless steel, chromium-cobalt alloys, or titanium alloys.

Another and/or alternative non-limiting object of the present disclosure is the provision of a medical device that includes the rhenium-chromium metal alloys or rhenium alloys wherein the medical device better conforms to an irregularly shaped body passageway when expanded in the body passageway as compared to a medical device formed by stainless steel, chromium-cobalt alloys, or titanium alloys.

Another and/or alternative non-limiting object of the present disclosure is the provision of a medical device that includes the rhenium-chromium metal alloys or rhenium alloys wherein the medical device has improved fatigue ductility when subjected to cold-working as compared to the cold-working of stainless steel, chromium-cobalt alloys, or titanium alloys.

Another and/or alternative non-limiting object of the present disclosure is the provision of a medical device that includes the rhenium-chromium metal alloys or rhenium alloys wherein the medical device has improved durability compared to stainless steel, chromium-cobalt alloys, or titanium alloys.

Another and/or alternative non-limiting object of the present disclosure is the provision of a medical device that includes the rhenium-chromium metal alloys or rhenium alloys wherein the medical device has improved hydrophilicity compared to stainless steel, chromium-cobalt alloys, or titanium alloys.

Another and/or alternative non-limiting object of the present disclosure is the provision of a medical device that includes the rhenium-chromium metal alloys or rhenium alloys wherein the medical device has reduced ion release in the body passageway compared to stainless steel, chromium-cobalt alloys, or titanium alloys.

Another and/or alternative non-limiting object of the present disclosure is the provision of a medical device that includes the rhenium-chromium metal alloys or rhenium alloys wherein the medical device is less of an irritant to the body than stainless steel or cobalt-chromium alloy or titanium alloys, thus can result in reduced inflammation, faster healing, and increased success rates of the medical device.

Another and/or alternative non-limiting object of the present disclosure is the provision of a medical device that includes the rhenium-chromium metal alloys or rhenium alloys wherein the rhenium-chromium metal alloys or rhenium alloys comprises rhenium and chromium; and

wherein a combined weight percent of the rhenium and chromium in the rhenium-chromium metal alloys or rhenium alloys is 95 wt. % to 99.999999 wt. %. and all values and ranges therebetween.

Another and/or alternative non-limiting object of the present disclosure is the provision of a medical device that includes the rhenium-chromium metal alloys or rhenium alloys wherein the rhenium-chromium metal alloys or rhenium alloys includes less than 0.1 wt. % metals and impurities.

Another and/or alternative non-limiting object of the present disclosure is the provision of a medical device that includes the rhenium-chromium metal alloys or rhenium alloys wherein the rhenium-chromium metal alloys or rhenium alloys has a controlled amount of nitrogen, oxygen, and carbon to reduce micro-cracking in the rhenium-chromium metal alloys or rhenium alloys, a nitrogen content in the rhenium-chromium metal alloys or rhenium alloys is less than a combined content of oxygen and carbon in the rhenium-chromium metal alloys or rhenium alloys, the rhenium-chromium metal alloys or rhenium alloys has an oxygen to nitrogen atomic ratio of at least about 1.2:1, the rhenium-chromium metal alloys or rhenium alloys has a carbon to nitrogen atomic ratio of at least about 2:1.

Another and/or alternative non-limiting object of the present disclosure is the provision of a medical device that includes the rhenium-chromium metal alloys or rhenium alloys; and wherein at least one region of the medical device includes at least one biological agent.

Another and/or alternative non-limiting object of the present disclosure is the provision of a medical device that includes the rhenium-chromium metal alloys or rhenium alloys; and wherein at least one region of the medical device includes at least one region of the medical device includes at least one polymer.

Another and/or alternative non-limiting object of the present disclosure is the provision of a medical device that includes the rhenium-chromium metal alloys or rhenium alloys; and wherein at least one region of the medical device includes at least one polymer, the at least one polymer at least partially coats, encapsulates, or combinations thereof at least one biological agent.

Another and/or alternative non-limiting object of the present disclosure is the provision of a medical device that includes the rhenium-chromium metal alloys or rhenium alloys; and wherein at least one region of the medical device includes at least one micro-structure on an outer surface of the medical device.

Another and/or alternative non-limiting object of the present disclosure is the provision of a medical device that includes the rhenium-chromium metal alloys or rhenium alloys; and wherein at least one region of the medical device includes at least one micro-structure on an outer surface of the medical device; and wherein the at least one microstructure is at least partially formed of, includes, or combinations thereof, a material consisting of a polymer, an agent, or combinations thereof.

Another and/or alternative non-limiting object of the present disclosure is the provision of a medical device that includes the rhenium-chromium metal alloys or rhenium alloys; and wherein the medical device includes an expandable frame.

Another and/or alternative non-limiting object of the present disclosure is the provision of a medical device that includes the rhenium-chromium metal alloys or rhenium alloys; and wherein the medical device includes an expandable frame; wherein the expandable frame includes a plurality of struts.

Another and/or alternative non-limiting object of the present disclosure is the provision of a medical device that includes the rhenium-chromium metal alloys or rhenium alloys; and wherein the medical device includes an expandable frame; wherein the expandable frame is configured to be crimped to a crimped state such that a maximum outer diameter of the expandable frame when in the crimped state is less than a maximum outer diameter of the expandable frame when fully expanded to an expanded state.

Another and/or alternative non-limiting object of the present disclosure is the provision of a medical device that includes the rhenium-chromium metal alloys or rhenium alloys; and wherein the medical device includes an expandable frame; wherein the expandable frame has a recoil of less than 5% after being subjected to a first crimping process.

Another and/or alternative non-limiting object of the present disclosure is the provision of a medical device that includes the rhenium-chromium metal alloys or rhenium alloys; and wherein the medical device includes an expandable frame; wherein the expandable frame has a recoil of less than 5% after being expanded from the crimped state to the expanded state.

Another and/or alternative non-limiting object of the present disclosure is the provision of a medical device that includes the rhenium-chromium metal alloys or rhenium alloys; and wherein the rhenium-chromium metal alloys or rhenium alloys has a hydrophilicity wherein a contact angle of a water droplet on a surface of the rhenium-chromium metal alloys or rhenium alloys of 25-45° (and all values and ranges therebetween).

Another and/or alternative non-limiting object of the present disclosure is the provision of a medical device that includes the rhenium-chromium metal alloys or rhenium alloys; and wherein the rhenium-chromium metal alloys or rhenium alloys has a maximum ion release of a primary component of the rhenium-chromium metal alloys or rhenium alloys when inserted or implanted on or in the body of the patient of no more than 0.5 μg/cm² per day; and wherein a primary component of the rhenium alloy is a metal in the rhenium alloy that constitutes at least 2 wt. % of the rhenium-chromium metal alloys or rhenium alloys.

Another and/or alternative non-limiting object of the present disclosure is the provision of a medical device that includes the rhenium-chromium metal alloys or rhenium alloys; and wherein the rhenium-chromium metal alloys or rhenium alloys has an absolute increase in ion release per dose of the rhenium-chromium metal alloys or rhenium alloys in tissue about the medical device of no more than 50 days after inserted or implanted on or in the body of a patient.

Another and/or alternative non-limiting object of the present disclosure is the provision of a medical device that includes the rhenium-chromium metal alloys or rhenium alloys; and wherein the rhenium-chromium metal alloys or rhenium alloys comprises rhenium and chromium and wherein A) a weight percent of the rhenium in the rhenium-chromium metal alloys or rhenium alloys is at least 50 wt. %; 0-25 wt. % alloying agent; a weight percent of the chromium in the rhenium-chromium metal alloys or rhenium alloys is at least 25 wt. %; a combined weight percent of the rhenium and the chromium is at least 75 wt. % of the rhenium-chromium metal alloys or rhenium alloys; a weight percent of the rhenium in the rhenium-chromium metal alloys or rhenium alloys is greater than a weight percent of the chromium in the rhenium-chromium metal alloys or rhenium alloys, or B) a weight percent of the rhenium in the rhenium-chromium metal alloys or rhenium alloys is less than 50 wt. %; the rhenium-chromium metal alloys or rhenium alloys including 0.1-50 wt. % alloying agent; alloying agent including one or more metals selected from a group consisting of molybdenum, bismuth, niobium, tantalum, titanium, vanadium, tungsten, manganese, zirconium, technetium, ruthenium, rhodium, hafnium, osmium, copper, yttrium, zirconium, and iridium; a weight percent of the chromium in the rhenium-chromium metal alloys or rhenium alloys is at least 25 wt. %; a combined weight percent of the rhenium and the chromium is at least 75 wt. % of the rhenium-chromium metal alloys or rhenium alloys.

Another and/or alternative non-limiting object of the present disclosure is the provision of a medical device that includes the rhenium-chromium metal alloys or rhenium alloys; and wherein the rhenium-chromium metal alloys or rhenium alloys wherein the weight percent of rhenium in the rhenium-chromium metal alloys or rhenium alloys is at least 60 wt. % and the weight percent of the chromium in the rhenium-chromium metal alloys or rhenium alloys is at least 30 wt. %.

Another and/or alternative non-limiting object of the present disclosure is the provision of a medical device that includes the rhenium-chromium metal alloys or rhenium alloys; and wherein the rhenium-chromium metal alloys or rhenium alloys wherein the weight percent of rhenium in the rhenium-chromium metal alloys or rhenium alloys is at least 64 wt. % and the weight percent of the chromium in the rhenium-chromium metal alloys or rhenium alloys is at least 32.5 wt. %.

Another and/or alternative non-limiting object of the present disclosure is the provision of a medical device that includes the rhenium-chromium metal alloys or rhenium alloys; and wherein the rhenium-chromium metal alloys or rhenium alloys wherein the alloying agent constitutes 0.1-25 wt. % of the rhenium-chromium metal alloys or rhenium alloys; the alloying agent including one or more metals selected from a group consisting of molybdenum, bismuth, niobium, tantalum, titanium, vanadium, tungsten, manganese, zirconium, technetium, ruthenium, rhodium, hafnium, osmium, copper, yttrium, zirconium, and iridium.

Another and/or alternative non-limiting object of the present disclosure is the provision of a medical device that includes the rhenium-chromium metal alloys or rhenium alloys; and wherein the rhenium-chromium metal alloys or rhenium alloys wherein the alloying agent constitutes 0.1-5 wt. % of the rhenium-chromium metal alloys or rhenium alloys.

Another and/or alternative non-limiting object of the present disclosure is the provision of a medical device that includes the rhenium-chromium metal alloys or rhenium alloys; and wherein the rhenium-chromium metal alloys or rhenium alloys wherein the rhenium-chromium metal alloys or rhenium alloys includes 0-0.1 wt. % impurities.

Another and/or alternative non-limiting object of the present disclosure is the provision of a medical device that includes the rhenium-chromium metal alloys or rhenium alloys; and wherein the rhenium-chromium metal alloys or rhenium alloys wherein the rhenium-chromium metal alloys or rhenium alloys has a controlled amount of nitrogen, oxygen, and carbon to reduce micro-cracking in the rhenium-chromium metal alloys or rhenium alloys, a nitrogen content in the rhenium-chromium metal alloys or rhenium alloys is less than a combined content of oxygen and carbon in the rhenium-chromium metal alloys or rhenium alloys, the rhenium-chromium metal alloys or rhenium alloys has an oxygen to nitrogen atomic ratio of at least about 1.2:1, the rhenium-chromium metal alloys or rhenium alloys has a carbon to nitrogen atomic ratio of at least about 2:1.

Another and/or alternative non-limiting object of the present disclosure is the provision of a medical device that includes the rhenium-chromium metal alloys or rhenium alloys; and wherein the medical device includes at least one biological agent and/or at least one polymer.

Another and/or alternative non-limiting object of the present disclosure is the provision of a medical device that includes the rhenium-chromium metal alloys or rhenium alloys; and wherein the medical device includes at least one micro-structure on an outer surface of the medical device.

Another and/or alternative non-limiting object of the present disclosure is the provision of a medical device that includes the rhenium-chromium metal alloys or rhenium alloys; and wherein the medical device includes an expandable frame formed of the rhenium-chromium metal alloys or rhenium alloys; the expandable frame including a plurality of struts; the expandable frame is configured to be crimped to a crimped state such that a maximum outer diameter of the expandable frame when in the crimped state is less than a maximum outer diameter of the expandable frame when fully expanded to an expanded state.

Another and/or alternative non-limiting object of the present disclosure is the provision of a medical device that includes the rhenium-chromium metal alloys or rhenium alloys; and wherein the medical device is an expandable stent or an expandable prosthetic heart valve.

Another and/or alternative non-limiting object of the present disclosure is the provision of a method for forming a rod, a tube, a green part, a near net part, a medical device and the like that is at least partially formed of the rhenium-chromium metal alloys or rhenium alloys by a forming process selected from the group consisting of a sintering process, a cold isostatic pressing (CIP) process and a hot isostatic pressing (HIP) process; an average particle size of metal particles used in the forming process is less than about 150 microns; the metal particles having a purity of at least 99%; the rhenium-chromium metal alloys or rhenium alloys comprising rhenium and chromium wherein A) a weight percent of the rhenium in the rhenium-chromium metal alloys or rhenium alloys is at least 50 wt. %; 0-25 wt. % alloying agent; a weight percent of the chromium in the rhenium-chromium metal alloys or rhenium alloys is at least 25 wt. %; a combined weight percent of the rhenium and the chromium is at least 75 wt. % of the rhenium-chromium metal alloys or rhenium alloys; a weight percent of the rhenium in the rhenium-chromium metal alloys or rhenium alloys is greater than a weight percent of the chromium in the rhenium-chromium metal alloys or rhenium alloys, or B) a weight percent of the rhenium in the rhenium-chromium metal alloys or rhenium alloys is less than 50 wt. %; the rhenium-chromium metal alloys or rhenium alloys including 0.1-50 wt. % alloying agent; alloying agent including one or more metals selected from a group consisting of molybdenum, bismuth, niobium, tantalum, titanium, vanadium, tungsten, manganese, zirconium, technetium, ruthenium, rhodium, hafnium, osmium, copper, yttrium, zirconium, and iridium; a weight percent of the chromium in the rhenium-chromium metal alloys or rhenium alloys is at least 25 wt. %; a combined weight percent of the rhenium and the chromium is at least 75 wt. % of the rhenium-chromium metal alloys or rhenium alloys.

Another and/or alternative non-limiting object of the present disclosure is the provision of a method for forming a rod, a tube, a green part, a near net part, a medical device and the like wherein the forming process includes the sintering process; the sintering process occurs at a temperature of at least 1600° C.

Another and/or alternative non-limiting object of the present disclosure is the provision of a method for forming a rod, a tube, a green part, a near net part, a medical device and the like wherein the sintering process occurs in a non-oxidizing environment.

Another and/or alternative non-limiting object of the present disclosure is the provision of a method for forming a rod, a tube, a green part, a near net part, a medical device and the like wherein the sintering process occur under a vacuum or under pressures exceeding 1 atm.

Another and/or alternative non-limiting object of the present disclosure is the provision of a method for forming a rod, a tube, a green part, a near net part, a medical device and the like wherein the sintering process is for about 2-40 hours.

Another and/or alternative non-limiting object of the present disclosure is the provision of a method for forming a rod, a tube, a green part, a near net part, a medical device and the like wherein the forming process includes the hot isostatic pressing (HIP) process, the hot isostatic pressing (HIP) process occurs at a temperature of at least 1700° C.

Another and/or alternative non-limiting object of the present disclosure is the provision of a method for forming a rod, a tube, a green part, a near net part, a medical device and the like wherein the hot isostatic pressing (HIP) process occurs in a non-oxidizing environment.

Another and/or alternative non-limiting object of the present disclosure is the provision of a method for forming a rod, a tube, a green part, a near net part, a medical device and the like wherein the hot isostatic pressing (HIP) process occurs under a vacuum or under pressures exceeding 1 atm.

Another and/or alternative non-limiting object of the present disclosure is the provision of a method for forming a rod, a tube, a green part, a near net part, a medical device and the like wherein the formed rod, tube, green part, near net part, medical device and the like is subjected to cold working.

Another and/or alternative non-limiting object of the present disclosure is the provision of a method for forming a rod, a tube, a green part, a near net part, a medical device and the like wherein the formed rod, tube, green part, near net part, medical device and the like is preheated to at least 1200° C. for at least 5 minutes prior to subjecting the formed rod, tube, green part, near net part, medical device and the like to a rolling process and/or a swaging process.

Another and/or alternative non-limiting object of the present disclosure is the provision of a rhenium based alloy containing 10-60 atomic weight percent (atw. %) Re and containing one or more metals selected from the group consisting of Mo, Cr, Ta, Nb, Ti, and Zr.

Another and/or alternative non-limiting object of the present disclosure is the provision of a rhenium based alloy comprising 0.5-50 atw. % Re and Cr 0.5-70 atw. %.

Another and/or alternative non-limiting object of the present disclosure is the provision of a rhenium based alloy is formed by powder pressing and sintering at a temperature from 1000-2500° C. from 5-32 hours to achieve densification of the powder pressed starting material in a non-oxidizing environment of hydrogen, argon, hydrogen and argon, or a vacuum.

Another and/or alternative non-limiting object of the present disclosure is the provision of a rhenium based alloy undergoes hot isostatic pressing from 1000-2500° C. in a hydrogen, argon, or hydrogen and argon environment under a pressure of 50-500 Mpa.

Another and/or alternative non-limiting object of the present disclosure is the provision of a rhenium based alloy undergoes a reduction process requiring a pre-heat from 800-2500° C. for 5-40 minutes.

Another and/or alternative non-limiting object of the present disclosure is the provision of a rhenium based alloy that has a crystalline structure of a body-centered cubic (BCC).

Another and/or alternative non-limiting object of the present disclosure is the provision of a rhenium based alloy wherein an elongation of the rhenium metal alloy is 5-45%.

Another and/or alternative non-limiting object of the present disclosure is the provision of a rhenium based alloy wherein a reduction in area of the rhenium metal alloy formation and/or post processing of the rhenium metal alloy is 35-80%.

Another and/or alternative non-limiting object of the present disclosure is the provision of a rhenium based alloy wherein a yield strength of the rhenium metal alloy is from 600-1800 Mpa.

Another and/or alternative non-limiting object of the present disclosure is the provision of a rhenium based alloy wherein an ultimate strength of the rhenium metal alloy is 600-2000 Mpa.

These and other advantages will become apparent to those skilled in the art upon the reading and following of this description.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

In one non-limiting object of the present disclosure, there is provided the rhenium-chromium metal alloys or rhenium alloys.

In another non-limiting object of the present disclosure, there is provided the rhenium-chromium metal alloys or rhenium alloys wherein a weight percent of rhenium in the rhenium-chromium metal alloys or rhenium alloys is at least 50 wt. %; a weight percent of chromium in the rhenium-chromium metal alloys or rhenium alloys is at least 25 wt. %.

In another non-limiting object of the present disclosure, there is provided the rhenium-chromium metal alloys or rhenium alloys, and wherein the rhenium-chromium metal alloys or rhenium alloys has a controlled amount of nitrogen, oxygen, and carbon to reduce micro-cracking in the rhenium-chromium metal alloys or rhenium alloys, a nitrogen content in the rhenium-chromium metal alloys or rhenium alloys is less than a combined content of oxygen and carbon in the rhenium-chromium metal alloys or rhenium alloys, the rhenium-chromium metal alloys or rhenium alloys has an oxygen to nitrogen atomic ratio of at least about 1.2:1, the rhenium-chromium metal alloys or rhenium alloys has a carbon to nitrogen atomic ratio of at least about 2:1.

In another non-limiting object of the present disclosure, there is provided the rhenium-chromium metal alloys or rhenium alloys, and wherein the rhenium-chromium metal alloys or rhenium alloys is used to at least partially form a medical device.

In another non-limiting object of the present disclosure, there is provided the rhenium-chromium metal alloys or rhenium alloys, and wherein the rhenium-chromium metal alloys or rhenium alloys is used to at least partially form a medical device; and wherein at least one region of the medical device includes at least one biological agent.

In another non-limiting object of the present disclosure, there is provided the rhenium-chromium metal alloys or rhenium alloys, and wherein the rhenium-chromium metal alloys or rhenium alloys is used to at least partially form a medical device; and wherein at least one region of the medical device includes at least one polymer.

In another non-limiting object of the present disclosure, there is provided the rhenium-chromium metal alloys or rhenium alloys, and wherein the rhenium-chromium metal alloys or rhenium alloys is used to at least partially form a medical device; and wherein at least one region of the medical device includes at least one polymer, the at least one polymer at least partially coats, encapsulates, or combinations thereof at least one biological agent.

In another non-limiting object of the present disclosure, there is provided the rhenium-chromium metal alloys or rhenium alloys, and wherein the rhenium-chromium metal alloys or rhenium alloys is used to at least partially form a medical device; and wherein at least one micro-structure is located on an outer surface of the medical device; and wherein the at least one microstructure optionally is at least partially formed of, includes, or combinations thereof, a material consisting of a polymer, an agent, or combinations thereof.

In another non-limiting object of the present disclosure, there is provided the rhenium-chromium metal alloys or rhenium alloys, and wherein the rhenium-chromium metal alloys or rhenium alloys is used to at least partially form a medical device; and wherein the medical device includes an expandable frame formed of the rhenium-chromium metal alloys or rhenium alloys; the expandable frame including a plurality of struts; the expandable frame is optionally configured to be crimped to a crimped state such that a maximum outer diameter of the expandable frame when in the crimped state is less than a maximum outer diameter of the expandable frame when fully expanded to an expanded state; and wherein the expandable frame optionally has a recoil of less than 5% (e.g., 0.1-4.99 and all values and ranges therebetween) after being subjected to a first crimping process; and wherein the expandable frame optionally has a recoil of less than 5% (e.g., 0.1-4.99 and all values and ranges therebetween) after being expanded from the crimped state to the expanded state; and wherein the rhenium-chromium metal alloys or rhenium alloys optionally has a hydrophilicity wherein a contact angle of a water droplet on a surface of the rhenium-chromium metal alloys or rhenium alloys of 25-45° (e.g., 0.1-4.99 and all values and ranges therebetween); and wherein the rhenium-chromium metal alloys or rhenium alloys optionally has a maximum ion release of a primary component of the rhenium-chromium metal alloys or rhenium alloys when inserted or implanted on or in the body of the patient of no more than 0.5 μg/cm² per day (e.g., 0.001-0.5 μg/cm² per day and all values and ranges therebetween); and wherein the primary component constitutes at least 2 wt. % of the rhenium-chromium metal alloys or rhenium alloys; and wherein the rhenium-chromium metal alloys or rhenium alloys optionally has an absolute increase in ion release per dose of rhenium-chromium metal alloy in tissue about the medical device of no more than 50 days after inserted or implanted on or in the body of a patient.

In another non-limiting object of the present disclosure, there is provided the rhenium-chromium metal alloys or rhenium alloys, and wherein the rhenium-chromium metal alloys or rhenium alloys is used to at least partially form a medical device; and wherein the medical device is an expandable stent or an expandable prosthetic heart valve.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be the to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

1-22. (canceled)

23. A method for forming component in the form of a rod, a tube, a green part, a near net part, or all or a portion of a medical device, and wherein said component is at least partially formed of a rhenium-chromium metal alloy; said method comprises:
a. providing a metal powder; said metal powder has a particle size of an average particle size of metal particles used in said forming process is less than about 150 microns; said metal powder includes rhenium and chromium;
b. pressing said metal powder together to form an intermediate component; said step of pressing includes one or more of cold isostatic pressing (CIP) and/or hot isostatic pressing (HIP); said step of pressing occurring at a pressure of 50-500 MPa; said step of pressing occurring in a hydrogen environment, argon environment, hydrogen and argon environment, or other type of non-oxidizing environment;
c. sintering said intermediate component to form a sintered intermediate component; said step of sintering occurring at under a vacuum or under pressures exceeding 1 atm; said step of sintering occurring at a sintering temperature is 1550° C.-1850° C.; said step of sintering occurring for 2-40 hours; said step of sintering occurring in a non-oxidizing environment;
d. swaging said sintered intermediate component to shape and/or reduce one or dimensions of said sintered intermediate component to partially or fully form said component; said step of swaging occurring at a temperature of 10° C.-400° C.; said step of swaging occurring in an inert or non-reducing environment; and
wherein said rhenium-chromium metal alloy has a crystalline structure of body-centered cubic (BCC); and
wherein said rhenium-chromium metal alloy comprises:
A) at least 50 wt. % of said rhenium; at least 25 wt. % of said chromium; 0-25 wt. % alloying agent; a combined weight percent of said rhenium and said chromium is at least 75 wt. % of said rhenium-chromium metal alloy; a weight percent of said rhenium in said rhenium-chromium metal alloy is greater than a weight percent of said chromium in said rhenium-chromium metal alloy; said alloying agent includes one or more metals selected from a group consisting of molybdenum, bismuth, niobium, tantalum, titanium, vanadium, tungsten, manganese, zirconium, technetium, ruthenium, rhodium, hafnium, osmium, copper, yttrium, zirconium, and iridium; or
B) less than 50 wt. % of said rhenium; 0.1-50 wt. % alloying agent; said alloying agent includes one or more metals selected from a group consisting of molybdenum, bismuth, niobium, tantalum, titanium, vanadium, tungsten, manganese, zirconium, technetium, ruthenium, rhodium, hafnium, osmium, copper, yttrium, zirconium, and iridium; a weight percent of said chromium in said rhenium-chromium metal alloy is at least 25 wt. %; a combined weight percent of said rhenium and said chromium is at least 50 wt. % of said rhenium-chromium metal alloy; or
C) 50-75 wt. % rhenium, 25-50 wt. % Cr, and 0.5-25 wt. % of said alloying agent; said alloying agent includes one or more metals selected from the group consisting of bismuth, iridium, manganese, molybdenum, niobium, tantalum, vanadium, titanium, tungsten, yttrium, and zirconium; or
D) 50-75 wt. % rhenium, 25-50 wt. % Cr, and 0.5-25 wt. % of said alloying agent; said alloying agent includes one or more metals selected from the group consisting of bismuth, iridium, molybdenum, niobium, tantalum, vanadium, yttrium, and zirconium; and said metal alloy includes 0-0.1 wt. % of secondary materials; said secondary materials are selected from the group consisting of a) metals other than rhenium, bismuth, iridium, molybdenum, niobium, tantalum, vanadium, yttrium, and zirconium, b) carbon, c) oxygen and d) nitrogen; or
E) 55-75 wt. % rhenium, 25-45 wt. % Cr, and 0.5-25 wt. % of said alloying agent; said alloying agent includes one or more metals selected from the group consisting of bismuth, iridium, molybdenum, niobium, tantalum, vanadium, yttrium, and zirconium; and said metal alloy includes 0-0.1 wt. % of secondary materials; said secondary materials are selected from the group consisting of a) metals other than rhenium, bismuth, iridium, molybdenum, niobium, tantalum, vanadium, yttrium, and zirconium, b) carbon, c) oxygen and d) nitrogen; or
F) 50-75 wt. % rhenium, 25-50 wt. % Cr, and 0.5-25 wt. % of said alloying agent; said alloying agent includes one or more metals selected from the group consisting of bismuth, iridium, manganese, molybdenum, niobium, tantalum, vanadium, titanium, tungsten, yttrium, and zirconium; or
G) 50-75 wt. % rhenium, 25-50 wt. % Cr, and 0.5-25 wt. % of said alloying agent; said alloying agent includes one or more metals selected from the group consisting of bismuth, iridium, molybdenum, niobium, tantalum, vanadium, yttrium, and zirconium; and said metal alloy includes 0-0.1 wt. % of secondary materials; said secondary materials are selected from the group consisting of a) metals other than rhenium, bismuth, iridium, molybdenum, niobium, tantalum, vanadium, yttrium, and zirconium, b) carbon, c) oxygen and d) nitrogen; or
H) 55-75 wt. % rhenium, 25-45 wt. % Cr, and 0.5-25 wt. % of said alloying agent; said alloying agent includes one or more metals selected from the group consisting of bismuth, iridium, molybdenum, niobium, tantalum, vanadium, yttrium, and zirconium; and said metal alloy includes 0-0.1 wt. % of secondary materials; said secondary materials are selected from the group consisting of a) metals other than rhenium, bismuth, iridium, molybdenum, niobium, tantalum, vanadium, yttrium, and zirconium, b) carbon, c) oxygen and d) nitrogen; or
I. 0.5-60 atomic weight percent (atw. %) Re and 0.5-70 awt. % chromium; and one or more of molybdenum, tantalum, niobium, titanium and zirconium; a combined atomic weight percent of rhenium and chromium is at least 60 awt. %.

24. The method as defined in claim 23, wherein said weight percent of rhenium in said rhenium-chromium metal alloy is greater than 60 wt. %; said weight percent of said chromium in said rhenium-chromium metal alloy is at least 30 wt. %.

25. The method as defined in claim 23, wherein said alloying agent constitutes 0.1-5 wt. % of said rhenium-chromium metal alloy; said alloying agent includes one or more metals selected from a group consisting of bismuth, iridium, molybdenum, niobium, tantalum, vanadium, yttrium, and zirconium.

26. The method as defined in claim 24, wherein said alloying agent constitutes 0.1-5 wt. % of said rhenium-chromium metal alloy; said alloying agent includes one or more metals selected from a group consisting of bismuth, iridium, molybdenum, niobium, tantalum, vanadium, yttrium, and zirconium.

27. The method as defined in claim 23, further including the step of nitriding an outer surface of said component.

28. The method as defined in claim 24, further including the step of nitriding an outer surface of said component.

29. The method as defined in claim 26, further including the step of nitriding an outer surface of said component.

30. The method as defined in claim 23, wherein said component is a rod, tube, expandable stent, expandable frame or hypotube.

31. The method as defined in claim 24, wherein said component is a rod, tube, expandable stent, expandable frame or hypotube.

32. The method as defined in claim 26, wherein said component is a rod, tube, expandable stent, expandable frame or hypotube.

33. The method as defined in claim 29, wherein said component is a rod, tube, expandable stent, expandable frame or hypotube.

34. The method as defined in claim 30, wherein said component is said expandable stent or said expandable frame; said component includes a plurality of struts; said component is configured to be crimped to a crimped state such that a maximum outer diameter of said component when in said crimped state is less than a maximum outer diameter of said component when fully expanded to an expanded state.

35. The method as defined in claim 32, wherein said component is said expandable stent or said expandable frame; said component includes a plurality of struts; said component is configured to be crimped to a crimped state such that a maximum outer diameter of said component when in said crimped state is less than a maximum outer diameter of said component when fully expanded to an expanded state.

36. The method as defined in claim 34, wherein said component has a recoil of a) less than 5% after being subjected to a first crimping process; and/or b) less than 5% after being expanded from said crimped state to said expanded state.

37. The method as defined in claim 35, wherein said component has a recoil of a) less than 5% after being subjected to a first crimping process; b) less than 5% after being expanded from said crimped state to said expanded state; c) has a hydrophilicity wherein a contact angle of a water droplet on a surface of said rhenium-chromium metal alloy of 25-45°; and d) said rhenium-chromium metal alloy has a maximum ion release of a primary component of said rhenium-chromium metal alloy when inserted or implanted on or in the body of the patient of no more than 0.5 μg/cm² per day, wherein said primary component constitutes at least 2 wt. % of said rhenium-chromium metal alloy; and e) said rhenium-chromium metal alloy has an absolute increase in ion release per dose of rhenium-chromium metal alloy in tissue about said rhenium-chromium metal alloy of no more than 50 days after inserted or implanted on or in the body of a patient.

38. The method as defined in claim 36, wherein said component has a recoil of a) less than 5% after being subjected to a first crimping process; b) less than 5% after being expanded from said crimped state to said expanded state; c) has a hydrophilicity wherein a contact angle of a water droplet on a surface of said rhenium-chromium metal alloy of 25-45°; and d) said rhenium-chromium metal alloy has a maximum ion release of a primary component of said rhenium-chromium metal alloy when inserted or implanted on or in the body of the patient of no more than 0.5 μg/cm² per day, wherein said primary component constitutes at least 2 wt. % of said rhenium-chromium metal alloy; and e) said rhenium-chromium metal alloy has an absolute increase in ion release per dose of rhenium-chromium metal alloy in tissue about said rhenium-chromium metal alloy of no more than 50 days after inserted or implanted on or in the body of a patient.
